(19) United States
(12) Patent Application Publication
(10) Pub. No.: US 2010/0005545 A1
(43) Pub. Date: Jan. 7, 2010

(54) Compositions and Methods of Using RNA Interference of SCA1-Like Genes for Control of Nematodes

(75) Inventor: Steve Hill, Cary, NC (US)

Correspondence Address: BASF CORPORATION, CARL-BOSCH-STRASSE 38, LUDWIGSHAFEN, D67056, DE

(73) Assignee: BASF Plant Science GmbH, Ludwigshafen (DE)

(21) Appl. No.: 12/524,729

(22) PCT Filed: Feb. 7, 2008
(86) PCT No.: PCT/EP2008/051480

§ 371 Date: Jul. 28, 2009

Related U.S. Application Data
(60) Provisional application No. 60/900,622, filed on Feb. 9, 2007.

(51) Int. Cl. A01H 5/00; A61K 31/7088; C07H 21/02; C12N 15/82

(52) U.S. Cl. 800/279; 514/44 R; 536/23.1; 800/301

(57) ABSTRACT

The present invention concerns double stranded RNA compositions and transgenic plants capable of inhibiting expression of essential genes in parasitic nematodes, and methods associated therewith. Specifically, the invention relates to the use of RNA interference to inhibit expression of a target essential nematode gene, which is a nematode sca1-like gene, and relates to the generation of plants that have increased resistance to parasitic nematodes.

### CROSS REFERENCE TO RELATED APPLICATIONS

This application claims the priority benefit of U.S. Provisional Application Ser. No. 60/900,622 filed Feb. 9, 2007.

### FIELD OF THE INVENTION

The field of this invention is the control of nematodes, in particular the control of soybean cyst nematodes. The invention also relates to the introduction of genetic material into plants that are susceptible to nematodes in order to increase resistance to nematodes.

### BACKGROUND OF THE INVENTION

Nematodes are microscopic wormlike animals that feed on the roots, leaves, and stems of more than 2,000 row crops, vegetables, fruits, and ornamental plants, causing an estimated $100 billion crop loss worldwide. One common type of nematode is the root-knot nematode (RKN), whose feeding causes the characteristic galls on roots. Other root-feeding nematodes are the cyst- and lesion-types, which are more host specific.

Nematodes are present throughout the United States, but are mostly a problem in warm, humid areas of the South and West, and in sandy soils. Soybean cyst nematode (SCN), Heterodera glycines, was first discovered in the United States in North Carolina in 1954. It is the most serious pest of soybean plants. Some areas are so heavily infested by SCN that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. However, nematodes, including SCN, can cause significant yield loss without obvious above-ground symptoms. In addition, roots infected with SCN are dwarfed or stunted. Nematode infestation can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions whereas other species can take as long as a year, or longer, to complete the life cycle. When temperature and moisture levels become adequate in the spring, worm-shaped juveniles hatch from eggs in the soil. These juveniles are the only life stage of the nematode that can infect soybean roots.

The life cycle of SCN has been the subject of many studies and therefore can be used as an example for understanding a nematode life cycle. After penetrating the soybean roots, SCN juveniles move through the root until they contact vascular tissue, where they stop and begin to feed. The nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As the nematodes feed, they swell and eventually female nematodes become so large that they break through the root tissue and are exposed on the surface of the root.

Male SCN nematodes, which are not swollen as adults, migrate out of the root into the soil and fertilize the lemon-shaped adult females. The males then die, while the females remain attached to the root system and continue to feed. The eggs in the swollen females begin developing, initially in a mass or egg sac outside the body, then later within the body cavity. Eventually the entire body cavity of the adult female is filled with eggs, and the female nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the cysts for several years.

Nematodes can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. The promoters of these plant target genes can then be used to direct the specific expression of detrimental proteins or enzymes, or the expression of antisense RNA to the target gene or to general cellular genes. The plant promoters may also be used to confer nematode resistance specifically at the feeding site by transforming the plant with a construct comprising the promoter of the plant target gene linked to a gene whose product induces lethality in the nematode after ingestion.

Recently, RNA interference (RNAi), also referred to as gene silencing, has been proposed as a method for controlling nematodes. When double-stranded RNA (dsRNA) corresponding essentially to the sequence of a target gene or mRNA is introduced into a cell, expression from the target gene is inhibited (See e.g., U.S. Pat. No. 6,506,559). U.S. Pat. No. 6,506,559 demonstrates the effectiveness of RNAi against known genes in Caenorhabditis elegans, but does not demonstrate the usefulness of RNAi for controlling plant parasitic nematodes.

Use of RNAi to target essential nematode genes has been proposed, for example, in PCT Publication WO 01/96584, WO 01/17654, US 2004/0098761, US 2005/0091713, US 2005/0188438, US 2006/0037101, US 2006/0080749, US 2007/0199100, and US 2007/0250947.

A number of models have been proposed for the action of RNAi. In mammalian systems, dsRNAs larger than 30 nucleotides trigger induction of interferon synthesis and a global shut-down of protein syntheses, in a non-sequence-specific manner. However, U.S. Pat. No. 6,506,559 discloses that in nematodes, the length of the dsRNA corresponding to the target gene sequence may be at least 25, 50, 100, 200, 300, or 400 bases, and that even larger dsRNAs were also effective at inducing RNAi in C. elegans. It is known that when hairpin RNA constructs comprising double stranded regions ranging from 98 to 854 nucleotides were transformed into a number of plant species, the target plant genes were efficiently silenced. There is general agreement that in many organisms, including nematodes and plants, large pieces of dsRNA are cleaved into about 19-24 nucleotide fragments (siRNA) within cells, and that these siRNAs are the actual mediators of the RNAi phenomenon.

Although there have been numerous efforts to use RNAi to control plant parasitic nematodes, to date no transgenic nematode-resistant plant has been deregulated in any country. Accordingly, there continues to be a need to identify safe and effective compositions and methods for the controlling plant parasitic nematodes using RNAi, and for the production of plants having increased resistance to plant parasitic nematodes.

### SUMMARY OF THE INVENTION

The present inventors have discovered that down-regulation of the SCN gene CB377729, results in hindered development or death of SCN. The protein product of SCN gene CB377729 has highest homology to sarco-endoplasmic reticulum Ca⁺⁺ ATPases, or sca1-like genes (also known as SERCA pumps). In C. elegans the sca1 gene encodes a sarco-endoplasmic reticulum Ca⁺⁺ ATPase that is required for development and muscle function. Thus, the invention focuses on the elimination of plant parasitic nematodes using plant expressed dsRNAs that target plant parasitic nematode sca1 genes. The nucleic acids of the invention are capable of inhibiting expression of parasitic nematode target genes by RNA interference (RNAi). In accordance with the invention, the parasitic nematode target gene is a parasitic nematode sca1-like gene.

In one embodiment, the invention provides a dsRNA comprising (a) a first strand comprising a sequence substantially identical to a portion of a plant parasitic nematode sca1-like target gene; and (b) a second strand comprising a sequence substantially complementary to the first strand.

The invention is further embodied in a pool of dsRNA molecules comprising a multiplicity of RNA molecules each comprising a double stranded region having a length of about 19 to 24 nucleotides, wherein said RNA molecules are derived from a polynucleotide that is substantially identical to a portion of a plant parasitic nematode sca1-like gene.

In another embodiment, the invention provides a transgenic nematode-resistant plant capable of expressing a dsRNA that is substantially identical to a portion of a plant parasitic nematode sca1-like gene.

In another embodiment, the invention provides a transgenic plant capable of expressing a pool of dsRNA molecules, wherein each dsRNA molecule comprises a double stranded region having a length of about 19-24 nucleotides and wherein the RNA molecules are derived from a polynucleotide substantially identical to a portion of a plant parasitic nematode sca1-like gene.

In another embodiment, the invention provides a method of making a transgenic plant capable of expressing a pool of dsRNA molecules each of which is substantially identical to a portion of a plant parasitic nematode sca1-like gene in a plant, said method comprising the steps of: a) preparing a nucleic acid having a region that is substantially identical to a portion of the sca1-like gene, wherein the nucleic acid is able to form a double-stranded transcript of a portion of the sca1-like gene once expressed in the plant; b) transforming a recipient plant with said nucleic acid; c) producing one or more transgenic offspring of said recipient plant; and d) selecting the offspring for expression of said transcript.

The invention further provides a method of conferring nematode resistance to a plant, said method comprising the steps of: a) preparing a nucleic acid having a region that is substantially identical to a portion of a plant parasitic nematode sca1-like gene, wherein the nucleic acid is able to form a double-stranded transcript of a portion of the sca1-like gene once expressed in the plant; b) transforming a recipient plant with said nucleic acid; c) producing one or more transgenic offspring of said recipient plant; and d) selecting the offspring for nematode resistance.

The invention further provides an expression cassette and an expression vector comprising a sequence substantially identical to a portion of a plant parasitic nematode sca1-like gene.

### BRIEF DESCRIPTION OF THE DRAWINGS

FIG. 1a-1b shows the cDNA sequence of H. glycines sca1-like gene, which is identified as SEQ ID NO:1.

FIG. 2 provides the sets of primers that were used to isolate the H. glycines sca1-like gene (SEQ ID NOs:2-7) and C. elegans homologs of the H. glycines sca1-like gene (SEQ ID NOs:8-9) by PCR. FIG. 2 also shows a table containing the common primers that can be utilized in sequence isolation, including SL1 (SEQ ID NO: 13) and GeneRacer Oligo dT (SEQ ID NO: 12).

FIG. 3 shows the sequence of the C. elegans sca1-like gene fragment (SEQ ID NO:10) used in the RNAi feeding assay of Example 2.

FIG. 4 shows the sequence of the 499 nucleotide fragment (SEQ ID NO:11) used in the binary vector p(R)SA006 useful for transformation of soybean cells to produce the dsRNA of the invention in soybean plants, thereby inhibiting the H. glycines sca1-like target genes identified herein.

FIGS. 5a-5r show various 21 mers possible in SEQ ID NO. 1 by nucleotide position.

### DETAILED DESCRIPTION OF THE PREFERRED EMBODIMENTS

The present invention may be understood more readily by reference to the following detailed description of the preferred embodiments of the invention and the examples included herein. Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5^{th} Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized It is to be understood that the terminology used herein is for the purpose of describing specific embodiments only and is not intended to be limiting.

Throughout this application, various patent and literature publications are referenced. The disclosures of all of these publications and those references cited within those publications in their entireties are hereby incorporated by reference into this application in order to more fully describe the state of the art to which this invention pertains.

A “plant parasitic nematode sca1-like gene” or “sca1-like gene” is defined herein as a gene having at least 70% sequence identity to a polynucleotide comprising a sequence as set forth in SEQ ID NO:1, 10 or 11. Additional sca1-like genes (sca1-like gene homologs) may be isolated from nematodes other than SCN using the information provided herein and techniques known to those of skill in the art of biotechnology. For example, a nucleic acid molecule from a plant parasitic nematode that hybridizes under stringent conditions to the nucleic acid of SEQ ID NO:1 can be isolated from plant parasitic nematode cDNA libraries. Alternatively, mRNA can be isolated from nematodes (e.g., by the guanidinium-thiocyanate extraction procedure of Chirgwin et al., 1979, Biochemistry 18:5294-5299), and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon the nucleotide sequence shown in SEQ ID NO:1. Nucleic acid molecules corresponding to the sca1-like target genes defined herein can be amplified using cDNA or, alternatively, genomic DNA, as a template and appropriate oligonucleotide primers according to standard PCR amplification techniques. The nucleic acid molecules so amplified can be cloned into appropriate vectors and characterized by DNA sequence analysis.

As used herein, “RNAi” or “RNA interference” refers to the process of sequence-specific post-transcriptional gene silencing in nematodes, mediated by double-stranded RNA (dsRNA). As used herein, “dsRNA” refers to RNA that is partially or completely double stranded. Double stranded RNA is also referred to as small or short interfering RNA (siRNA), short interfering nucleic acid (siNA), short interfering RNA, micro-RNA (miRNA), and the like. In the RNAi process, dsRNA comprising a first strand that is substantially identical to a portion of a target gene, e.g. a sca1-like gene, and a second strand that is complementary to the first strand is introduced into a nematode, preferably by soaking and more preferably by feeding. After introduction into the nematode, the target gene-specific dsRNA is processed into relatively small fragments (siRNAs) and can subsequently become distributed throughout the nematode, leading to a loss-of-function mutation having a phenotype that, over the period of a generation, may come to closely resemble the phenotype arising from a complete or partial deletion of the target gene. Alternatively, the target gene-specific dsRNA is processed into relatively small fragments by a plant cell containing the RNAi processing machinery; and when the plant-processed small dsRNA is ingested by a parasitic nematode, the loss-of-function phenotype is obtained.

As used herein, taking into consideration the substitution of uracil for thymine when comparing RNA and DNA sequences, the term “substantially identical” as applied to dsRNA means that the nucleotide sequence of one strand of the dsRNA is at least about 80%-90% identical to 20 or more contiguous nucleotides of the target gene, more preferably, at least about 90-95% identical to 20 or more contiguous nucleotides of the target gene, and most preferably at least about 95%, 96%, 97%, 98% or 99% identical or absolutely identical to 20 or more contiguous nucleotides of the target gene. 20 or more nucleotides means a portion, being at least about 20, 21, 22, 23, 24, 25, 50, 100, 200, 300, 400, 500, 1000, 1500, consecutive bases or up to the full length of the target gene.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. As used herein, the term “substantially complementary” means that two nucleic acid sequences are complementary over at least at 80% of their nucleotides. Preferably, the two nucleic acid sequences are complementary over at least at 85%, 90%, 95%, 96%, 97%, 98%, 99% or more or all of their nucleotides. Alternatively, “substantially complementary” means that two nucleic acid sequences can hybridize under high stringency conditions. As used herein, the term “substantially identical” or “corresponding to” means that two nucleic acid sequences have at least 80% sequence identity. Preferably, the two nucleic acid sequences have at least 85%, 90%, 95%, 96%, 97%, 98%, 99% or 100% of sequence identity.

Also as used herein, the terms “nucleic acid” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the terms “contacting” and “administering” are used interchangeably, and refer to a process by which dsRNA of the present invention is delivered to a cell of a parasitic nematode, in order to inhibit expression of an essential target gene in the nematode. The dsRNA may be administered in a number of ways, including, but not limited to, direct introduction into a cell (i.e., intracellularly); or extracellular introduction into a cavity, interstitial space, or into the circulation of the nematode, oral introduction, the dsRNA may be introduced by bathing the nematode in a solution containing dsRNA, or the dsRNA may be present in food source. Methods for oral introduction include direct mixing of dsRNA with food of the nematode, as well as engineered approaches in which a species that is used as food is engineered to express a dsRNA, then fed to the organism to be affected. For example, the dsRNA may be sprayed onto a plant, or the dsRNA may be applied to soil in the vicinity of roots, taken up by the plant and/or the parasitic nematode, or a plant may be genetically engineered to express the dsRNA in an amount sufficient to kill some or all of the parasitic nematode to which the plant is exposed.

As used herein, the term “control,” when used in the context of an infection, refers to the reduction or prevention of an infection. Reducing or preventing an infection by a nematode will cause a plant to have increased resistance to the nematode, however, such increased resistance does not imply that the plant necessarily has 100% resistance to infection. In preferred embodiments, the resistance to infection by a nematode in a resistant plant is greater than 10%, 20%, 30%, 40%, 50%, 60%, 70%, 80%, 90%, or 95% in comparison to a wild type plant that is not resistant to nematodes. Preferably the wild type plant is a plant of a similar, more preferably identical genotype as the plant having increased resistance to the nematode, but does not comprise a dsRNA directed to the target gene. The plant's resistance to infection by the nematode may be due to the death, sterility, arrest in development, or impaired mobility of the nematode upon exposure to the dsRNA specific to an essential gene. The term “resistant to nematode infection” or “a plant having nematode resistance” as used herein refers to the ability of a plant, as compared to a wild type plant, to avoid infection by nematodes, to kill nematodes or to hamper, reduce or stop the development, growth or multiplication of nematodes. This might be achieved by an active process, e.g. by producing a substance detrimental to the nematode, or by a passive process, like having a reduced nutritional value for the nematode or not developing structures induced by the nematode feeding site like syncytia or giant cells. The level of nematode resistance of a plant can be determined in various ways, e.g. by counting the nematodes being able to establish parasitism on that plant, or measuring development times of nematodes, proportion of male and female nematodes or, for cyst nematodes, counting the number of cysts or nematode eggs produced on roots of an infected plant or plant assay system.

The term “plant” is intended to encompass plants at any stage of maturity or development, as well as any tissues or organs (plant parts) taken or derived from any such plant unless otherwise clearly indicated by context. Plant parts include, but are not limited to, stems, roots, flowers, ovules, stamens, seeds, leaves, embryos, meristematic regions, callus tissue, anther cultures, gametophytes, sporophytes, pollen, microspores, protoplasts, hairy root cultures, and the like. The present invention also includes seeds produced by the plants of the present invention. In one embodiment, the seeds are true breeding for an increased resistance to nematode infection as compared to a wild-type variety of the plant seed. As used herein, a “plant cell” includes, but is not limited to, a protoplast, gamete producing cell, and a cell that regenerates into a whole plant. Tissue culture of various tissues of plants and regeneration of plants therefrom is well known in the art and is widely published.

As used herein, the term “transgenic” refers to any plant, plant cell, callus, plant tissue, or plant part that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

As used herein, the term “amount sufficient to inhibit expression” refers to a concentration or amount of the dsRNA that is sufficient to reduce levels or stability of mRNA or protein produced from a target gene in a parasitic nematode. As used herein, “inhibiting expression” refers to the absence or observable decrease in the level of protein and/or mRNA product from a target gene. Inhibition of target gene expression may be lethal to the parasitic nematode, or such inhibition may delay or prevent entry into a particular developmental step (e.g., metamorphosis), if plant disease is associated with a particular stage of the parasitic nematode's life cycle. The consequences of inhibition can be confirmed by examination of the outward properties of the nematode (as presented below in the examples).

In accordance with the invention, a parasitic nematode is contacted with a dsRNA, which specifically inhibits expression of a sca1-like target gene that is essential for survival, metamorphosis, or reproduction of the nematode. Preferably, the parasitic nematode comes into contact with the dsRNA after entering a plant that expresses the dsRNA. In one embodiment, the dsRNA is encoded by a vector that has been transformed into an ancestor of the infected plant.

In one embodiment, the parasitic nematode target gene is a homolog of the C. elegans sca1 gene, sca1-like was identified in screens for essential genes and phenotypic analyses indicate that loss of sca1-like activity results in embryonic and larval lethality. Example 2 below shows that feeding C. elegans RNAi molecules specific for the sca1 gene results in sterile adults, i.e., animals do not produce any progeny. Preferably it is a homolog of the C. elegans sca1 gene derived from a plant parasitic nematode. In this embodiment of the present invention, the parasitic nematode sca1 target gene comprises a sequence selected from the group consisting of: (a) the sequence set forth in SEQ ID NO:1, (b) a polynucleotide having at least 80% sequence identity to SEQ ID NO:1, 10 or 11; and (c) a polynucleotide from a parasitic nematode that hybridizes under stringent conditions to the sequence set forth in SEQ ID NO:1, 10 or 11.

Complete cDNAs corresponding to the sca1-like target gene of the invention may be isolated from parasitic nematodes other than H. glycines using the information provided herein and techniques known to those of skill in the art of biotechnology. For example, a nucleic acid molecule from a parasitic nematode that hybridizes under stringent conditions to a nucleotide sequence of SEQ ID NO:1, 10 or 11 can be isolated from parasitic nematode cDNA libraries. Alternatively, mRNA can be isolated from parasitic nematode cells, and cDNA can be prepared using reverse transcriptase (e.g., Moloney MLV reverse transcriptase. Synthetic oligonucleotide primers for polymerase chain reaction amplification can be designed based upon the nucleotide sequence shown in SEQ ID NO:1, 10 or 11. Examples for such primers are given by SEQ ID NO: 2, 3, 4, 5, 6, 7, 8, or 9. Nucleic acid molecules corresponding to the parasitic nematode target genes of the invention can be amplified using cDNA or, alternatively, genomic DNA, as a template and appropriate oligonucleotide primers according to standard PCR amplification techniques. The nucleic acid molecules so amplified can be cloned into appropriate vectors and characterized by DNA sequence analysis.

Accordingly, the dsRNA of the invention comprises a first strand that is substantially identical to a portion of the sca1-like target gene of a plant parasitic nematode genome and a second strand that is substantially complementary to the first strand. In preferred embodiments, the target gene is selected from the group consisting of: (a) a polynucleotide having the sequence set forth in SEQ ID NO:1, 10 or 11; (b) a polynucleotide having at least 80% sequence identity to SEQ ID NO:1, 10 or 11; and (c) a polynucleotide from a parasitic nematode that hybridizes under stringent conditions to a polynucleotide having the sequence set forth in SEQ ID NO:1, 10 or 11.

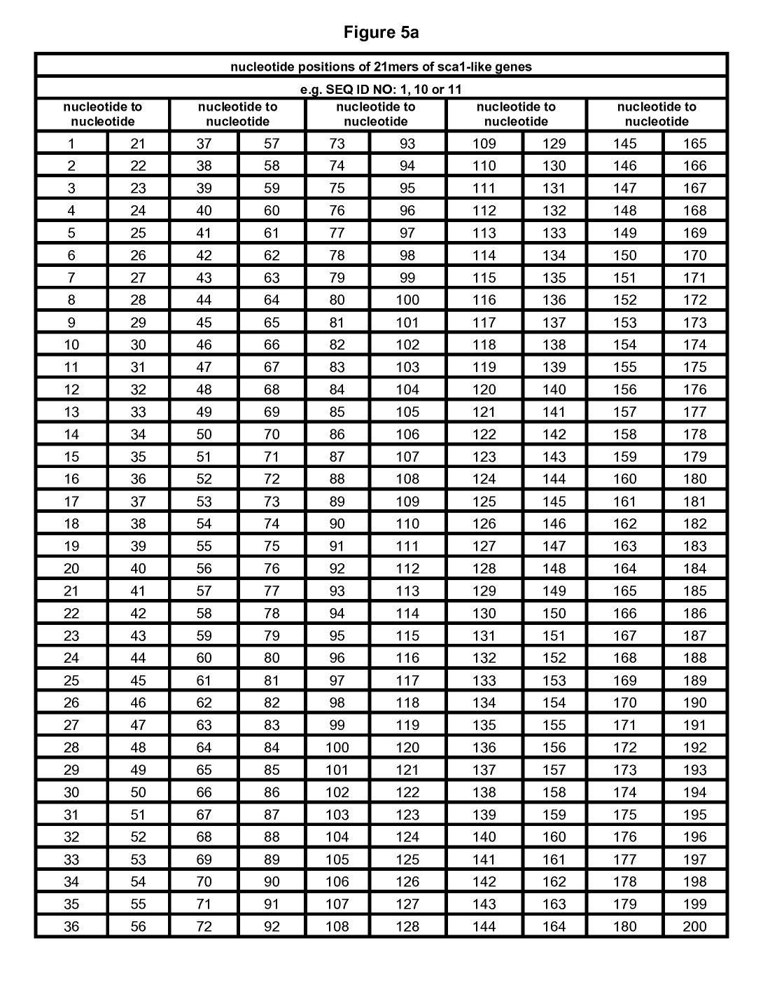

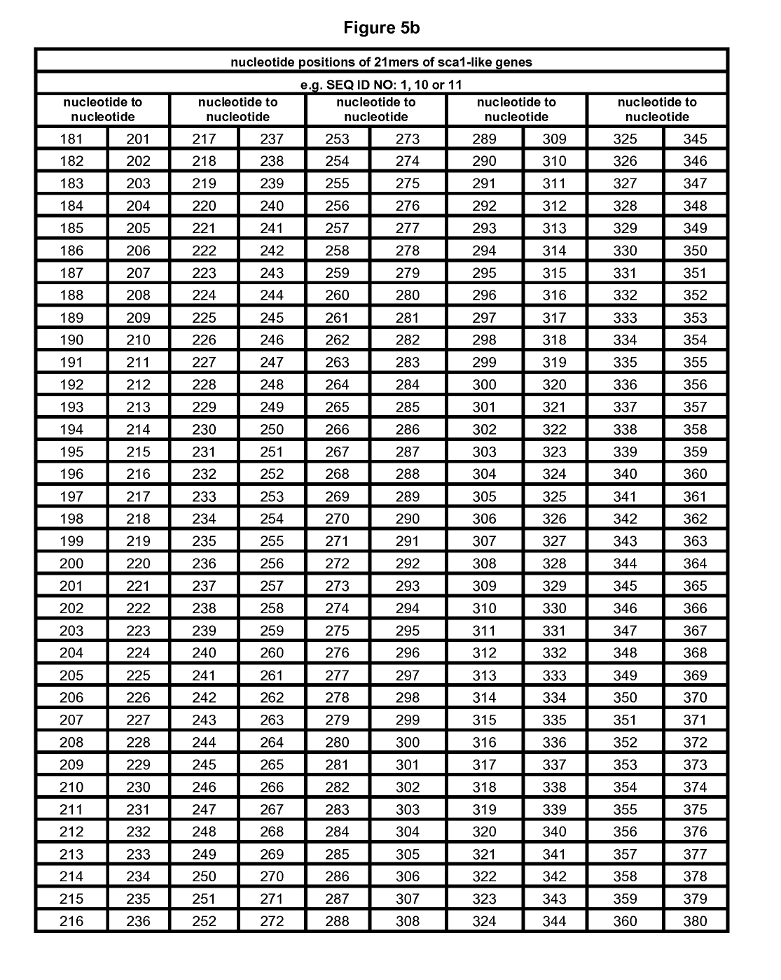

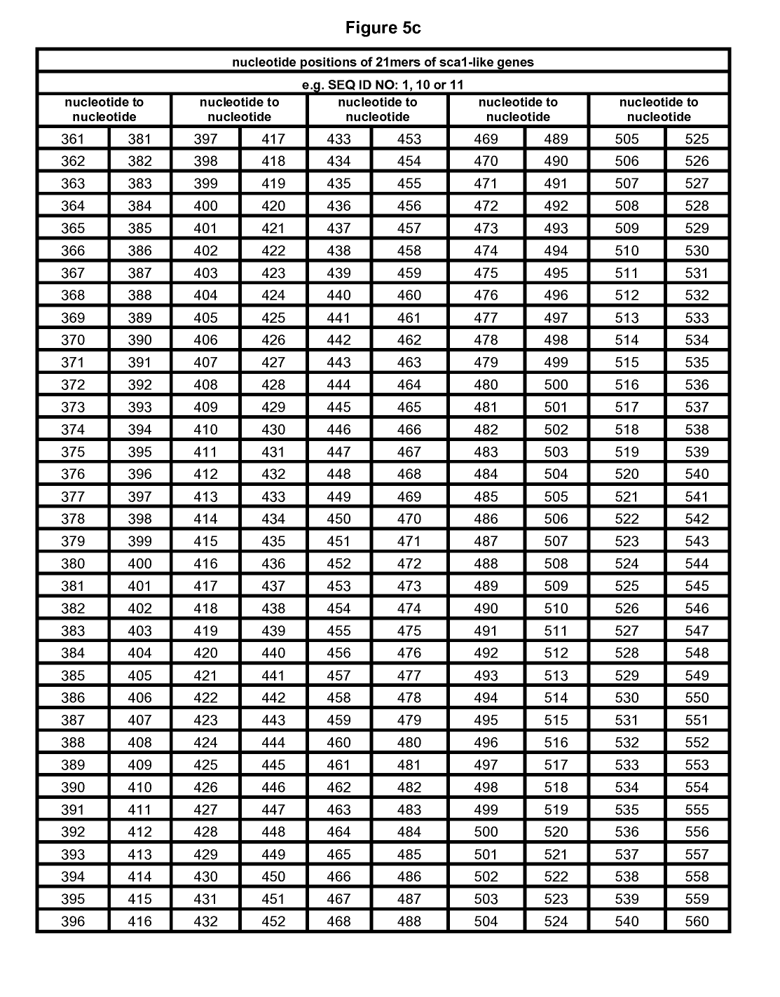

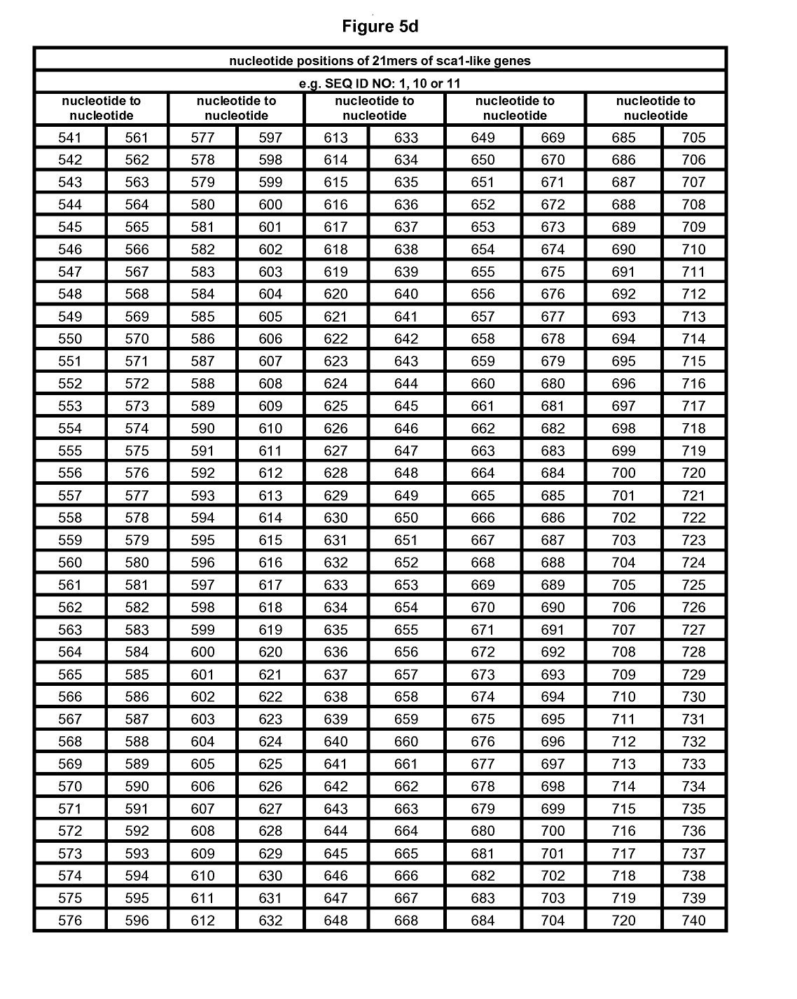

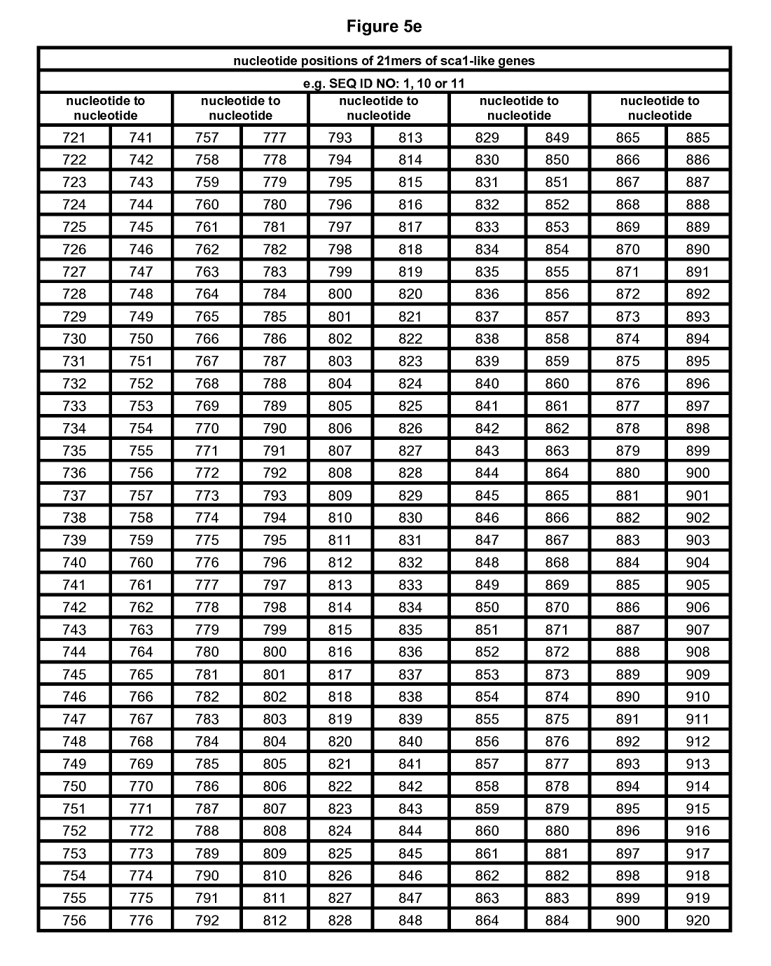

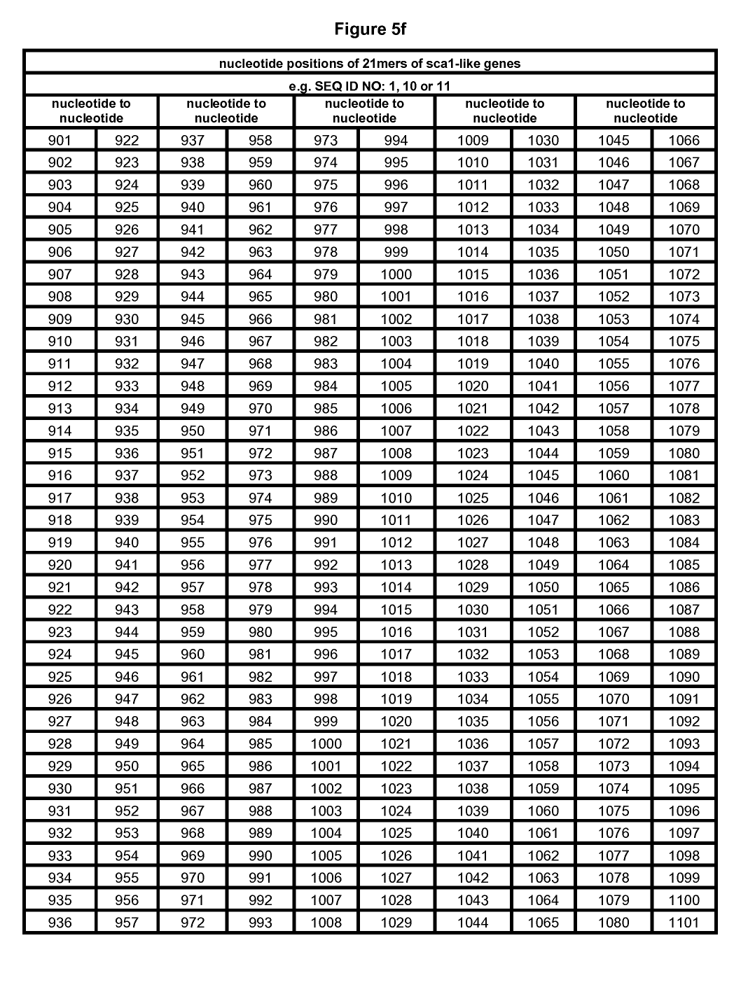

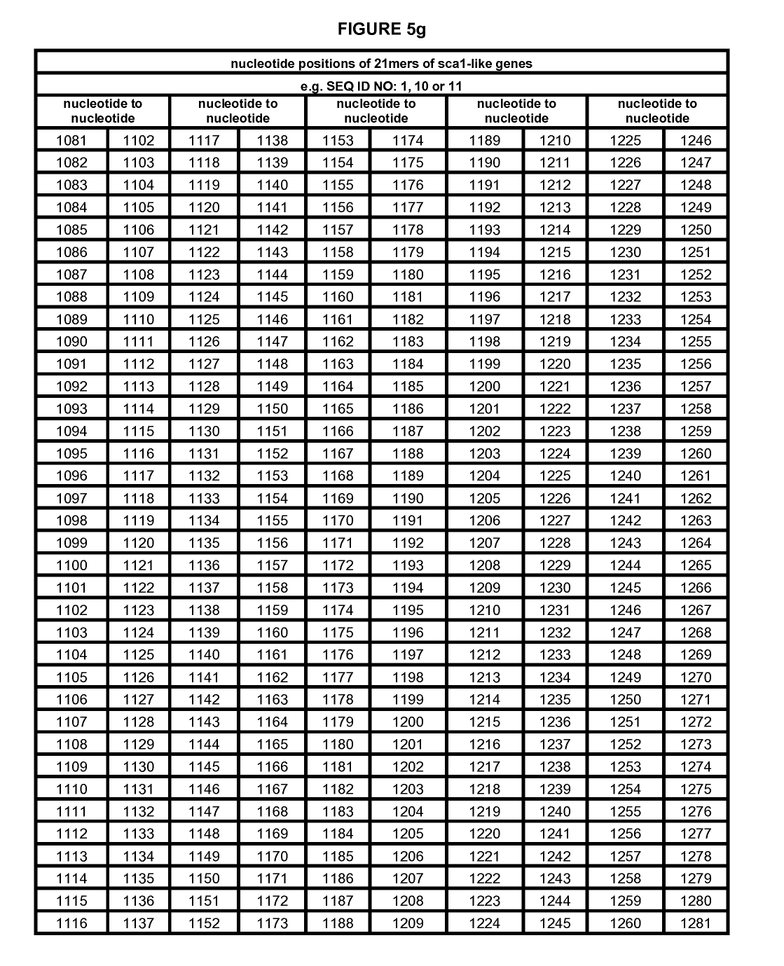

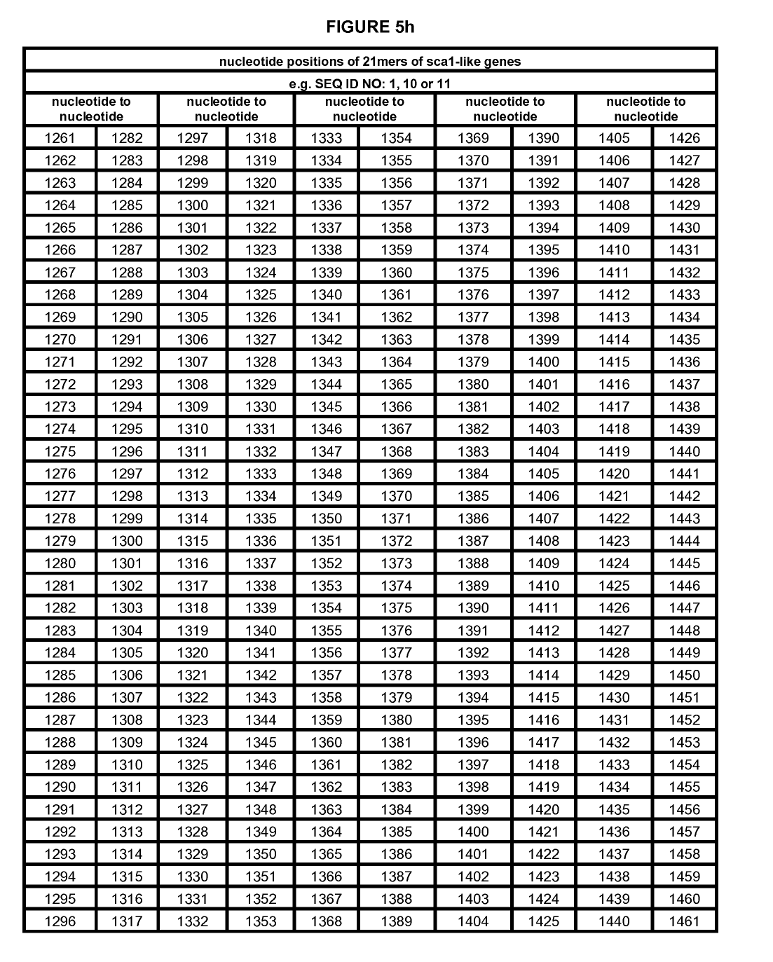

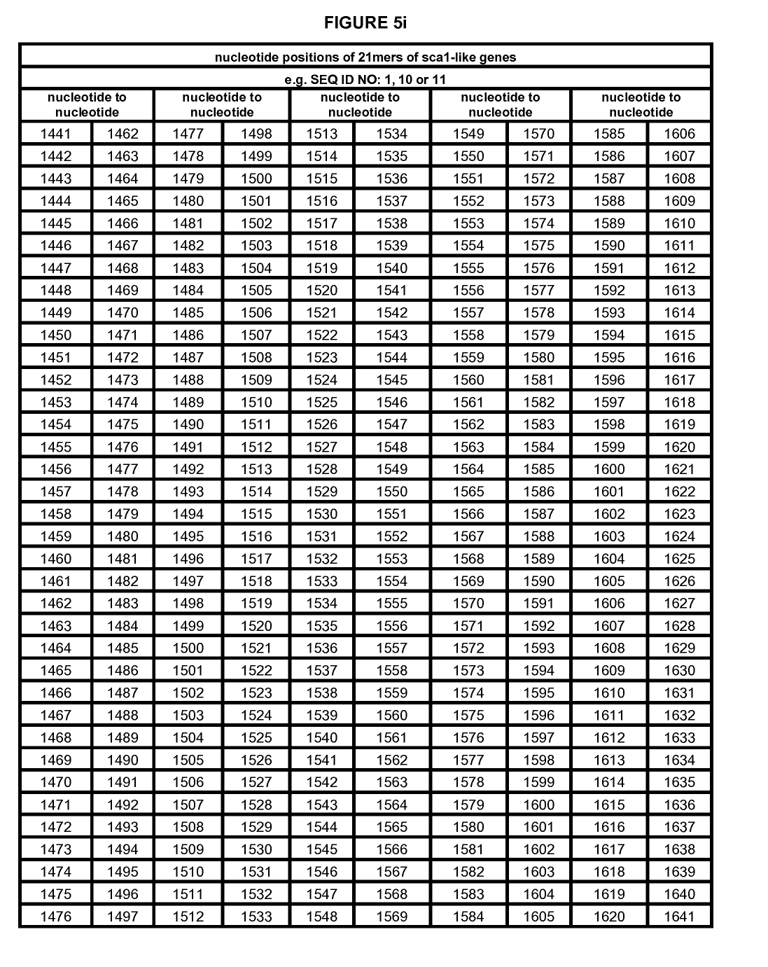

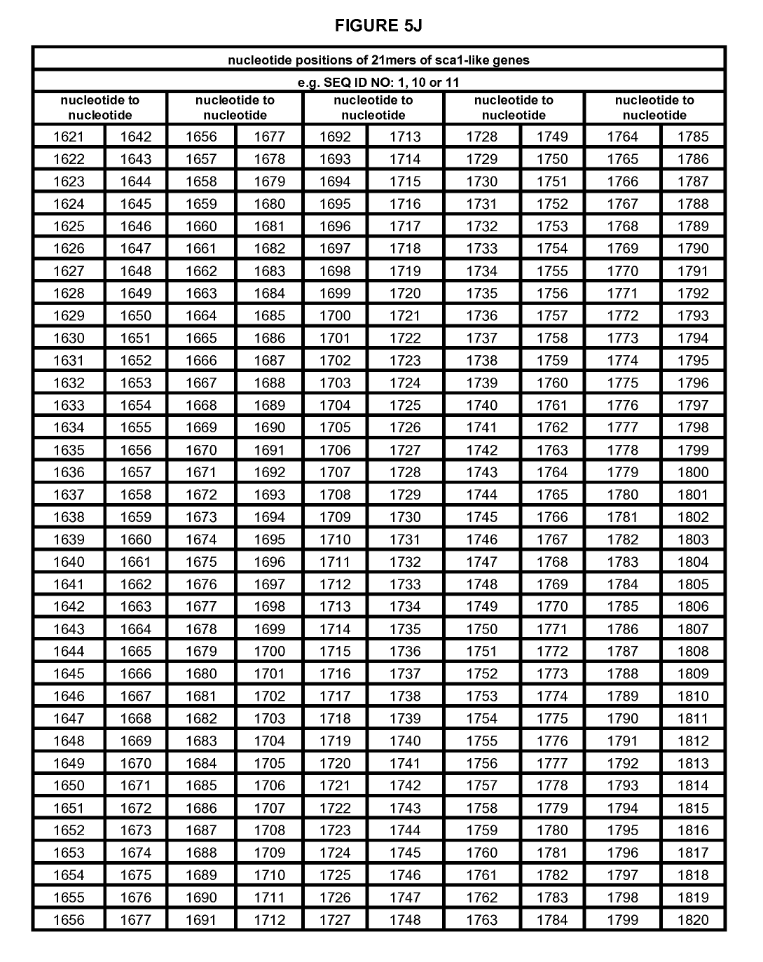

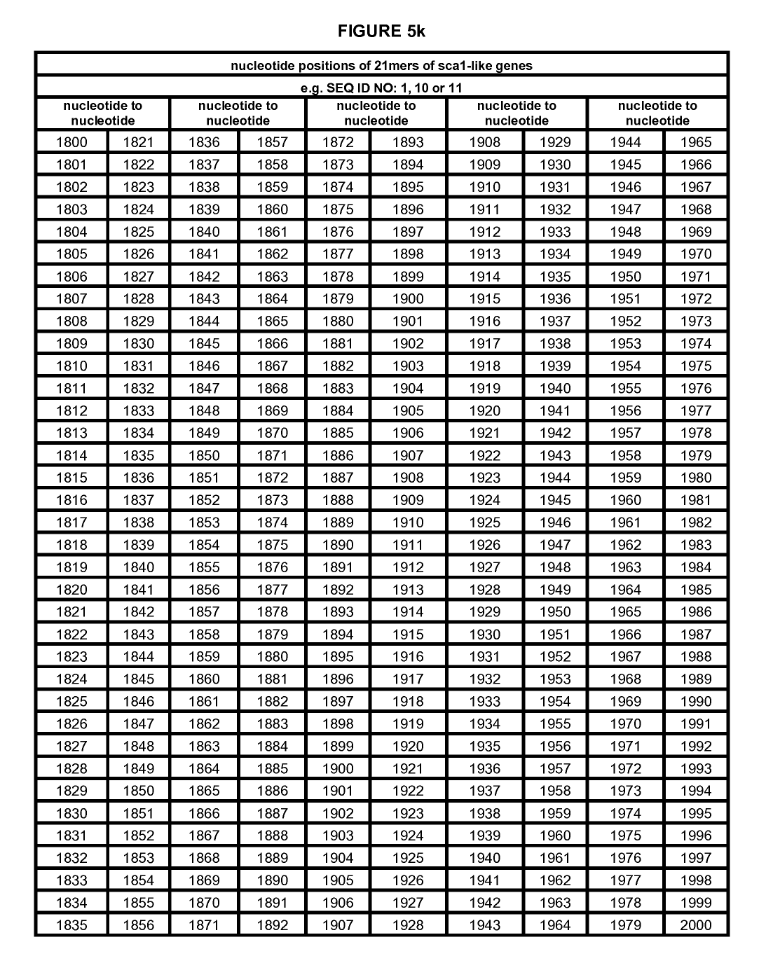

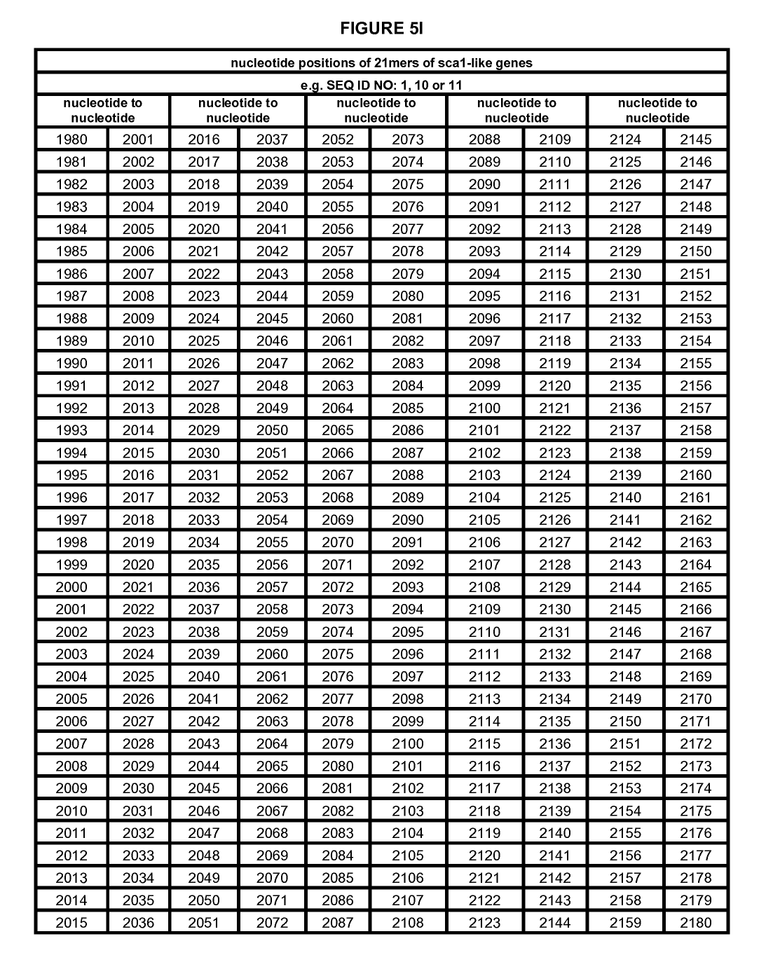

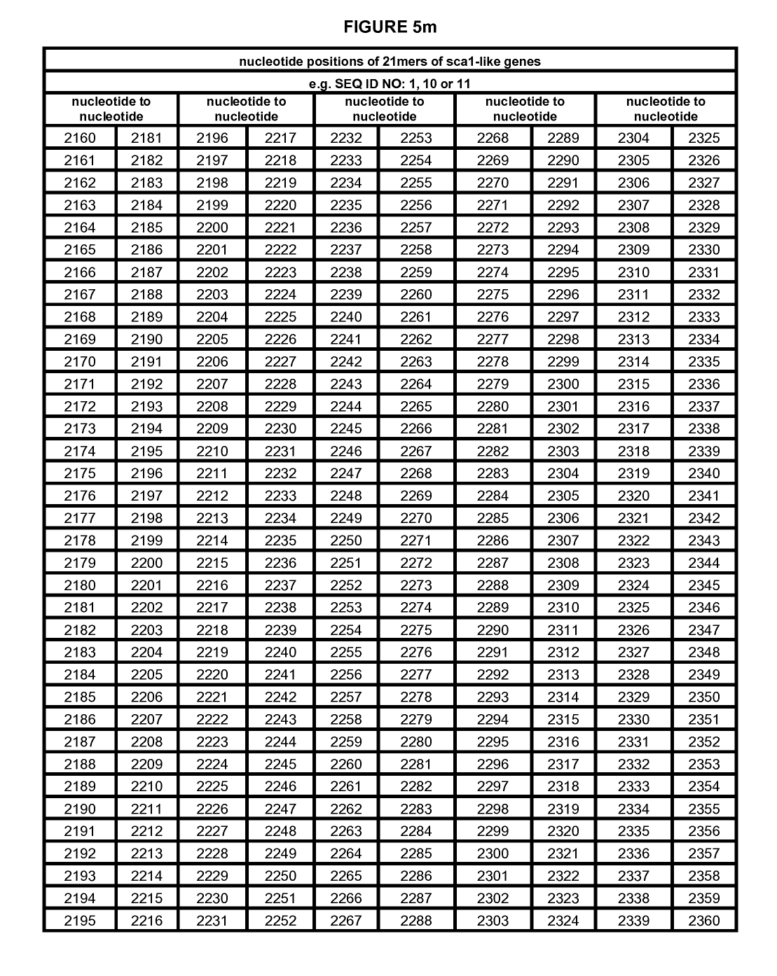

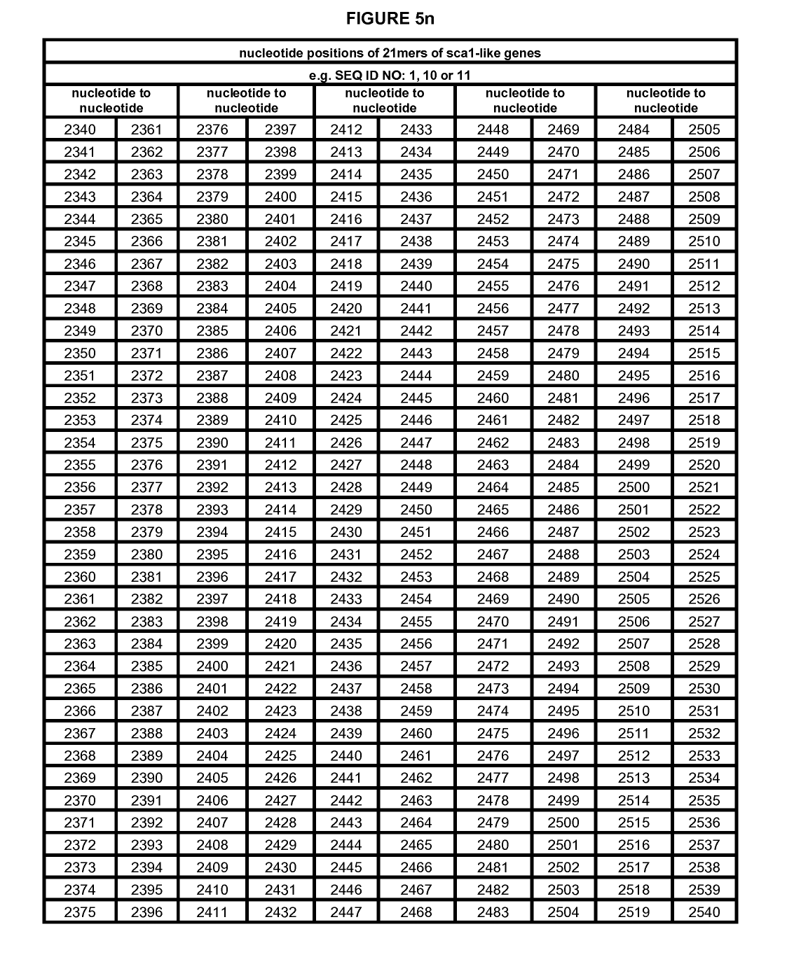

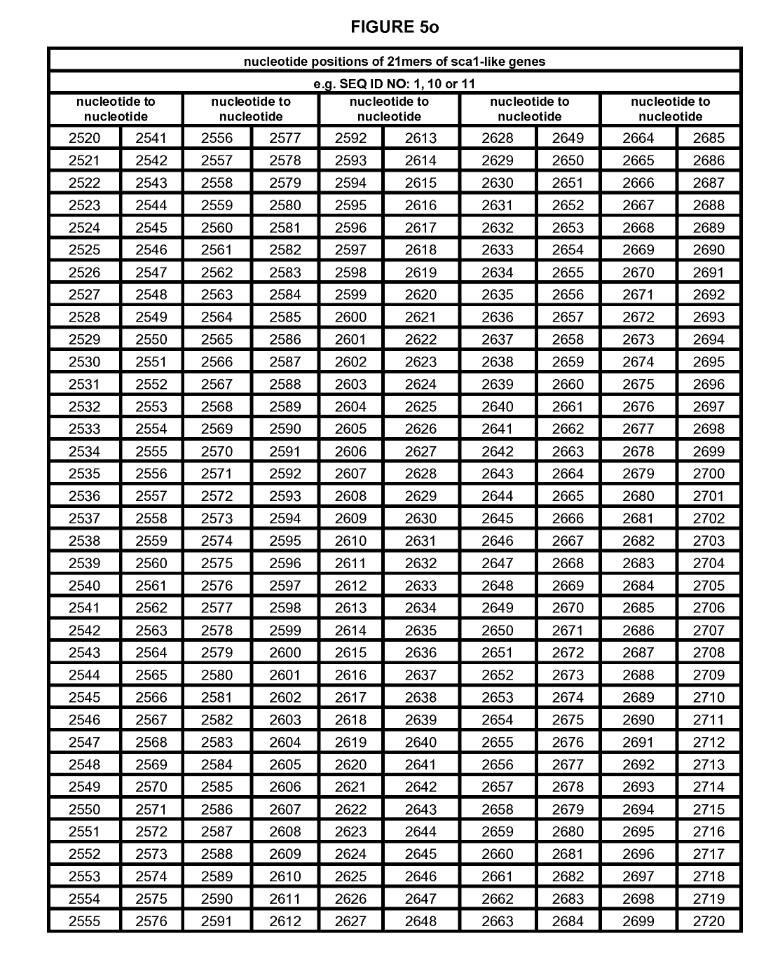

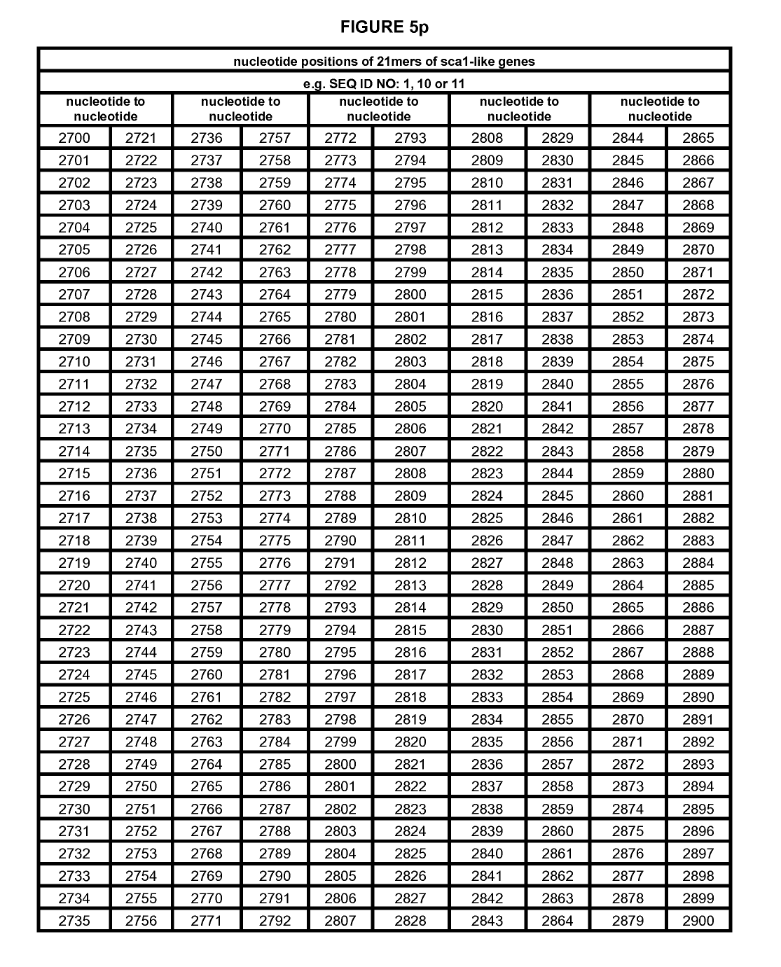

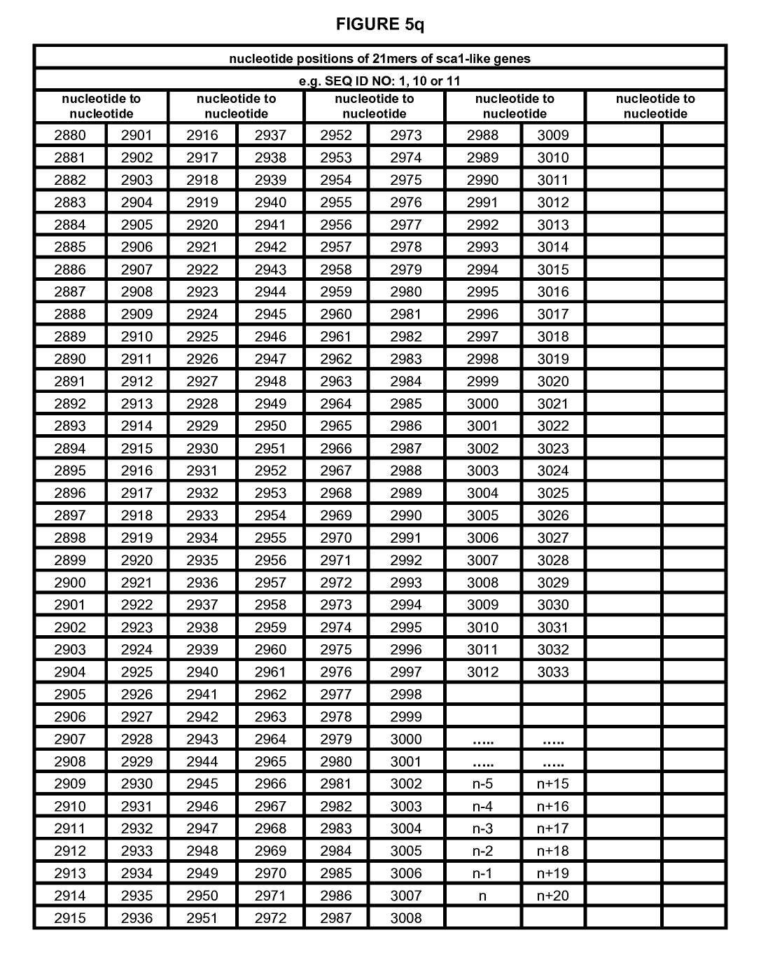

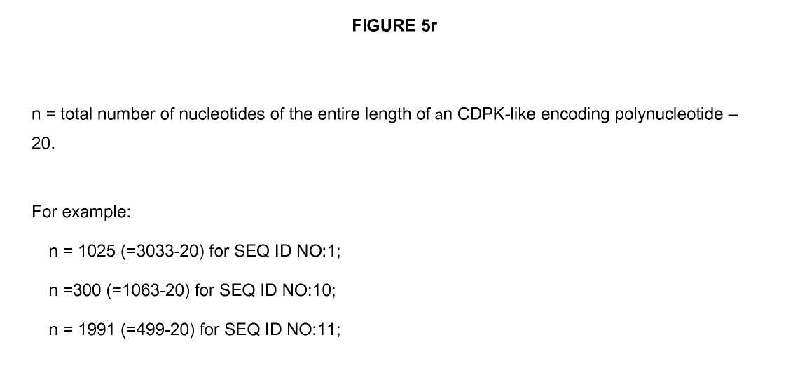

As discussed above, fragments of dsRNA larger than about 19-24 nucleotides in length are cleaved intracellularly by nematodes and plants to siRNAs of about 19-24 nucleotides in length, and these siRNAs are the actual mediators of the RNAi phenomenon. The table in FIGS. 5a-5r sets forth exemplary 21-mers of the SCN sca1-like gene from SCN, SEQ ID NO:1. This table can also be used to calculate the 19, 20, 22, 23, or 24-mers by adding or subtracting the appropriate number of nucleotides from each 21 mer. Thus the dsRNA of the present invention may range in length from about 19 nucleotides to about 500 consecutive nucleotides or up to the whole length of a sca1-like gene. Alternatively, the dsRNA of the invention has a length from about 21 nucleotides to about 600 consecutive nucleotides. Further, the dsRNA of the invention has a length from about 21 nucleotides to about 400 consecutive nucleotides, or from about 21 nucleotides to about 300 consecutive nucleotides.

As disclosed herein, 100% sequence identity between the dsRNA and the target gene is not required to practice the present invention. While a dsRNA comprising a nucleotide sequence identical to a portion of the sca1-like gene is preferred for inhibition, the invention can tolerate sequence variations that might be expected due to gene manipulation or synthesis, genetic mutation, strain polymorphism, or evolutionary divergence. Thus the dsRNAs of the invention also encompass dsRNAs comprising a mismatch with the target gene of at least 1, 2, or more nucleotides. For example, it is contemplated in the present invention that the 21 mer dsRNA sequences exemplified in FIGS. 7a-7j may contain an addition, deletion or substitution of 1, 2, or more nucleotides, so long as the resulting sequence still interferes with the sca1-like gene function.

Sequence identity between the dsRNAs of the invention and the sca1-like target genes may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 80% sequence identity, 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60° C. hybridization for 12-16 hours; followed by washing at 65° C. with 0.1% SDS and 0.1% SSC for about 15-60 minutes).

When dsRNA of the invention has a length longer than about 21 nucleotides, for example from about 50 nucleotides to about 1000 nucleotides, it will be cleaved randomly to dsRNAs of about 21 nucleotides within the plant or parasitic nematode cell, the siRNAs. The cleavage of a longer dsRNA of the invention will yield a pool of 21 mer dsRNAs, derived from the longer dsRNA. This pool of 21 mer dsRNAs is also encompassed within the scope of the present invention, whether generated intracellularly within the plant or nematode or synthetically using known methods of oligonucleotide synthesis.

The siRNAs of the invention have sequences corresponding to fragments of about 19-24 contiguous nucleotides across the entire sequence of the H. glycines sca1-like target gene. For example, a pool of siRNA of the invention derived from the H. glycines sca1-like gene as set forth in SEQ ID NO:1, 10 or 11 may comprise a multiplicity of RNA molecules which are selected from the group consisting of oligonucleotides substantially identical to the 21mer nucleotides of SEQ ID NO:1, 10 or 11 found in FIGS. 5a-5r. One of skill in the art would recognize that the siRNA can have a mismatch with the target gene of at least 1, 2, or more nucleotides. Further, these mismatches are intended to be included in the present invention. For example, it is contemplated in the present invention that the 21mer dsRNA sequences exemplified in FIGS. 5a-5r may contain an addition, deletion or substitution of 1, 2, or more nucleotides and the resulting sequence still interferes with the sca1-like gene function. A pool of siRNA of the invention derived from the H. glycines sca1-like target gene of SEQ ID NO:1, 10 or 11 may also comprise any combination of the specific RNA molecules having any of the 21 contiguous nucleotide sequences derived from SEQ ID NO:1, 10 or 11 set forth in FIGS. 5a-5r. Further, as multiple specialized Dicers in plants generate siRNAs typically ranging in size from 19 nt to 24 nt (See Henderson et al., 2006. Nature Genetics 38:721-725), the siRNAs of the present invention can may range from about 19 contiguous nucleotide sequences to about 24 contiguous nucleotide sequences. Similarly, a pool of siRNA of the invention may comprise a multiplicity of RNA molecules having any 19, 20, 21, 22, 23, or 24 contiguous nucleotide sequences derived from SEQ ID NO:1, 10 or 11. Alternatively, the pool of siRNA of the invention may comprise a multiplicity of RNA molecules having a combination of any 19, 20, 21, 22, 23, and/or 24 contiguous nucleotide sequences derived from SEQ ID NO:1, 10 or 11.

The dsRNA of the invention may optionally comprise a single stranded overhang at either or both ends. The double-stranded structure may be formed by a single selfcomplementary RNA strand (i.e. forming a hairpin loop) or two complementary RNA strands. RNA duplex formation may be initiated either inside or outside the cell. When the dsRNA of the invention forms a hairpin loop, it may optionally comprise an intron, as set forth in US 2003/0180945A1 or a nucleotide spacer, which is a stretch of sequence between the complementary RNA strands to stabilize the hairpin transgene in cells. Methods for making various dsRNA molecules are set forth, for example, in WO 99/53050 and in U.S. Pat. No. 6,506,559. The RNA may be introduced in an amount that allows delivery of at least one copy per cell. Higher doses of double-stranded material may yield more effective inhibition.

In another embodiment, the invention provides an isolated recombinant expression vector comprising a nucleic acid encoding a dsRNA molecule as described above, wherein expression of the vector in a host plant cell results in increased resistance to a parasitic nematode as compared to a wild-type variety of the host plant cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host plant cell into which they are introduced. Other vectors are integrated into the genome of a host plant cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., potato virus X, tobacco rattle virus, and Gemini virus), which serve equivalent functions.

The recombinant expression vectors of the invention comprise a nucleic acid of the invention in a form suitable for expression of the nucleic acid in a host plant cell, which means that the recombinant expression vector includes one or more regulatory sequences, e.g. promoters, selected on the basis of the host plant cells to be used for expression, which is operatively linked to the nucleic acid sequence to be expressed. With respect to a recombinant expression vector, the terms “operatively linked” and “in operative association” are interchangeable and are intended to mean that the nucleotide sequence of interest is linked to the regulatory sequence(s) in a manner which allows for expression of the nucleotide sequence (e.g., in a host plant cell when the vector is introduced into the host plant cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990) and Gruber and Crosby, in: Methods in Plant Molecular Biology and Biotechnology, Eds. Glick and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of dsRNA desired, etc. The expression vectors of the invention can be introduced into plant host cells to thereby produce dsRNA molecules of the invention encoded by nucleic acids as described herein.

In accordance with the invention, the recombinant expression vector comprises a regulatory sequence operatively linked to a nucleotide sequence that is a template for one or both strands of the dsRNA molecules of the invention. In one embodiment, the nucleic acid molecule further comprises a promoter flanking either end of the nucleic acid molecule, wherein the promoters drive expression of each individual DNA strand, thereby generating two complementary RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by 3 to 500 base or more pairs, and wherein after transcription, the RNA transcript folds on itself to form a hairpin. In accordance with the invention, the spacer region in the hairpin transcript may be any DNA fragment.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and be transiently expressed or transiently active. Whether present in an extra-chromosomal non-replicating vector or a vector that is integrated into a chromosome, the polynucleotide preferably resides in a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., 1984, EMBO J. 3:835) or functional equivalents thereof, but also all other terminators functionally active in plants are suitable. As plant gene expression is very often not limited on transcriptional levels, a plant expression cassette preferably contains other operatively linked sequences like translational enhancers such as the overdrive-sequence containing the 5′-untranslated leader sequence from tobacco mosaic virus enhancing the polypeptide per RNA ratio (Gallie et al., 1987, Nucl. Acids Research 15:8693-8711). Examples of plant expression vectors include those detailed in: Becker, D. et al., 1992, New plant binary vectors with selectable markers located proximal to the left border, Plant Mol. Biol. 20:1195-1197; Bevan, M. W., 1984, Binary Agrobacterium vectors for plant transformation, Nucl. Acid. Res. 12:8711-8721; and Vectors for Gene Transfer in Higher Plants; in: Transgenic Plants, Vol. 1, Engineering and Utilization, eds.: Kung and R. Wu, Academic Press, 1993, S. 15-38.

Plant gene expression should be operatively linked to an appropriate promoter conferring gene expression in a temporal-preferred, spatial-preferred, cell type-preferred, and/or tissue-preferred manner. Promoters useful in the expression cassettes of the invention include any promoter that is capable of initiating transcription in a plant cell present in the plant's roots. Such promoters include, but are not limited to those that can be obtained from plants, plant viruses and bacteria that contain genes that are expressed in plants, such as Agrobacterium and Rhizobium. Preferably, the expression cassette of the invention comprises a root-specific promoter, a pathogen inducible promoter, or a nematode inducible promoter. More preferably the nematode inducible promoter is or a parasitic nematode feeding site-specific promoter. A parasitic nematode feeding site-specific promoter may be specific for syncytial cells or giant cells or specific for both kinds of cells. A promoter is inducible, if its activity, measured on the amount of RNA produced under control of the promoter, is at least 30%, 40%, 50% preferably at least 60%, 70%, 80%, 90% more preferred at least 100%, 200%, 300% higher in its induced state, than in its un-induced state. A promoter is cell-, tissue- or organ-specific, if its activity, measured on the amount of RNA produced under control of the promoter, is at least 30%, 40%, 50% preferably at least 60%, 70%, 80%, 90% more preferred at least 100%, 200%, 300% higher in a particular cell-type, tissue or organ, then in other cell-types or tissues of the same plant, preferably the other cell-types or tissues are cell types or tissues of the same plant organ, e.g. a root. In the case of organ specific promoters, the promoter activity has to be compared to the promoter activity in other plant organs, e.g. leaves, stems, flowers or seeds.

The promoter may be constitutive, inducible, developmental stage-preferred, cell type-preferred, tissue-preferred or organ-preferred. Constitutive promoters are active under most conditions. Non-limiting examples of constitutive promoters include the CaMV 19S and 35S promoters (Odell et al., 1985, Nature 313:810-812), the sX CaMV 35S promoter (Kay et al., 1987, Science 236:1299-1302), the Sep1 promoter, the rice actin promoter (McElroy et al., 1990, Plant Cell 2:163-171), the Arabidopsis actin promoter, the ubiquitin promoter (Christensen et al., 1989, Plant Molec. Biol. 18:675-689); pEmu (Last et al., 1991, Theor. Appl. Genet. 81:581-588), the figwort mosaic virus 35S promoter, the Smas promoter (Velten et al., 1984, EMBO J. 3:2723-2730), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like. Promoters that express the dsRNA in a cell that is contacted by parasitic nematodes are preferred. Alternatively, the promoter may drive expression of the dsRNA in a plant tissue remote from the site of contact with the nematode, and the dsRNA may then be transported by the plant to a cell that is contacted by the parasitic nematode, in particular cells of, or close by nematode feeding sites, e.g. syncytial cells or giant cells.

Inducible promoters are active under certain environmental conditions, such as the presence or absence of a nutrient or metabolite, heat or cold, light, pathogen attack, anaerobic conditions, and the like. For example, the promoters TobRB7, AtRPE, AtPyk10, Gemini19, and AtHMG1 have been shown to be induced by nematodes (for a review of nematodeinducible promoters, see Ann. Rev. Phytopathol. (2002) 40:191-219; see also U.S. Pat. No. 6,593,513). Method for isolating additional promoters, which are inducible by nematodes are set forth in U.S. Pat. Nos. 5,589,622 and 5,824,876. Other inducible promoters include the hsp80 promoter from Brassica, being inducible by heat shock; the PPDK promoter is induced by light; the PR-1 promoter from tobacco, Arabidopsis, and maize are inducible by infection with a pathogen; and the Adh1 promoter is induced by hypoxia and cold stress. Plant gene expression can also be facilitated via an inducible promoter (For review, see Gatz, 1997, Annu. Rev. Plant Physiol. Plant Mol. Biol. 48:89-108). Chemically inducible promoters are especially suitable if time-specific gene expression is desired. Non-limiting examples of such promoters are a salicylic acid inducible promoter (PCT Application No. WO 95/19443), a tetracycline inducible promoter (Gatz et al., 1992, Plant J. 2:397-404) and an ethanol inducible promoter (PCT Application No. WO 93/21334).

Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stempreferred, root-preferred promoters and the like. Seed preferred promoters are preferentially expressed during seed development and/or germination. For example, seed preferred promoters can be embryo-preferred, endosperm preferred and seed coat-preferred. See Thompson et al., 1989, BioEssays 10:108. Examples of seed preferred promoters include, but are not limited to cellulose synthase (celA), Cim1, gamma-zein, globulin-1, maize 19 kD zein (cZ19B1) and the like.

Other suitable tissue-preferred or organ-preferred promoters include, but are not limited to, the napin-gene promoter from rapeseed (U.S. Pat. No. 5,608,152), the USP-promoter from Vicia faba (Baeumlein et al., 1991, Mol Gen Genet. 225(3):459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980), or the legumin B4 promoter (LeB4; Baeumlein et al., 1992, Plant Journal, 2(2):233-9), as well as promoters conferring seed specific expression in monocot plants like maize, barley, wheat, rye, rice, etc. Suitable promoters to note are the Ipt2 or Ipt1-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15 kD zein promoter, the 22 kD zein promoter, the 27 kD zein promoter, the g-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

In accordance with the present invention, the expression cassette comprises an expression control sequence operatively linked to a nucleotide sequence that is a template for one or both strands of the dsRNA. The dsRNA template comprises (a) a first stand having a sequence substantially identical to from about 19 to about 400-500, or up to the full length, consecutive nucleotides of SEQ ID NO:1; and (b) a second strand having a sequence substantially complementary to the first strand. In further embodiments, a promoter flanks either end of the template nucleotide sequence, wherein the promoters drive expression of each individual DNA strand, thereby generating two complementary RNAs that hybridize and form the dsRNA. In alternative embodiments, the nucleotide sequence is transcribed into both strands of the dsRNA on one transcription unit, wherein the sense strand is transcribed from the 5′ end of the transcription unit and the antisense strand is transcribed from the 3′ end, wherein the two strands are separated by about 3 to about 500 base pairs, and wherein after transcription, the RNA transcript folds on itself to form a hairpin.

In another embodiment, the vector contains a bidirectional promoter, driving expression of two nucleic acid molecules, whereby one nucleic acid molecule codes for the sequence substantially identical to a portion of a sca1-like gene and the other nucleic acid molecule codes for a second sequence being substantially complementary to the first strand and capable of forming a dsRNA, when both sequences are transcribed. A bidirectional promoter is a promoter capable of mediating expression in two directions.

In another embodiment, the vector contains two promoters one mediating transcription of the sequence substantially identical to a portion of a sca1-like gene and another promoter mediating transcription of a second sequence being substantially complementary to the first strand and capable of forming a dsRNA, when both sequences are transcribed. The second promoter might be a different promoter.

A different promoter means a promoter having a different activity in regard to cell or tissue specificity, or showing expression on different inducers for example, pathogens, abiotic stress or chemicals. For example, one promoter might by constitutive or tissue specific and another might be tissue specific or inducible by pathogens. In one embodiment one promoter mediates the transcription of one nucleic acid molecule suitable for over expression of a sca1-like gene, while another promoter mediates tissue- or cell-specific transcription or pathogen inducible expression of the complementary nucleic acid.

The invention is also embodied in a transgenic plant capable of expressing the dsRNA of the invention and thereby inhibiting the sca1-like genes in parasitic nematodes. The plant or transgenic plant may be any plant, such like, but not limited to trees, cut flowers, ornamentals, vegetables or crop plants. The plant may be from a genus selected from the group consisting of Medicago, Lycopersicon, Brassica, Cucumis, Solanum, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium, or the plant may be selected from a genus selected from the group consisting of Arabidopsis, Medicago, Lycopersicon, Brassica, Cucumis, Solanum, Juglans, Gossypium, Malus, Vitis, Antirrhinum, Brachipodium, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panicum, Pennisetum, Ranunculus, Senecio, Salpiglossis, Browaalia, Phaseolus, Avena, and Allium. In one embodiment the plant is a monocotyledonous plant or a dicotyledonous plant.

Preferably the plant is a crop plant. Crop plants are all plants, used in agriculture. Accordingly in one embodiment the plant is a monocotyledonous plant, preferably a plant of the family Poaceae, Musaceae, Liliaceae or Bromeliaceae, preferably of the family Poaceae. Accordingly, in yet another embodiment the plant is a Poaceae plant of the genus Zea, Triticum, Oryza, Hordeum, Secale, Avena, Saccharum, Sorghum, Pennisetum, Setaria, Panicum, Eleusine, Miscanthus, Brachypodium, Festuca or Lolium. When the plant is of the genus Zea, the preferred species is Z. mays. When the plant is of the genus Triticum, the preferred species is T. aestivum, T. speltae or T. durum. When the plant is of the genus Oryza, the preferred species is O. sativa. When the plant is of the genus Hordeum, the preferred species is H. vulgare. When the plant is of the genus Secale, the preferred species S. cereale. When the plant is of the genus Avena, the preferred species is A. sativa. When the plant is of the genus Saccarum, the preferred species is S. officinarum. When the plant is of the genus Sorghum, the preferred species is S. vulgare, S. bicolor or S. sudanense. When the plant is of the genus Pennisetum, the preferred species is P. glaucum. When the plant is of the genus Setaria, the preferred species is S. italica. When the plant is of the genus Panicum, the preferred species is P. miliaceum or P. virgatum. When the plant is of the genus Eleusine, the preferred species is E. coracana. When the plant is of the genus Miscanthus, the preferred species is M. sinensis. When the plant is a plant of the genus Festuca, the preferred species is F. arundinaria, F. rubra or F. pratensis. When the plant is of the genus Lolium, the preferred species is L. perenne or L. multiflorum. Alternatively, the plant may be Triticosecale.

Alternatively, in one embodiment the plant is a dicotyledonous plant, preferably a plant of the family Fabaceae, Solanaceae, Brassicaceae, Chenopodiaceae, Asteraceae, Malvaceae, Linacea, Euphorbiaceae, Convolvulaceae Rosaceae, Cucurbitaceae, Theaceae, Rubiaceae, Sterculiaceae or Citrus. In one embodiment the plant is a plant of the family Fabaceae, Solanaceae or Brassicaceae. Accordingly, in one embodiment the plant is of the family Fabaceae, preferably of the genus Glycine, Pisum, Arachis, Cicer, Vicia, Phaseolus, Lupinus, Medicago or Lens. Preferred species of the family Fabaceae are M. truncatula, M. sativa, G. max, P. sativum, A. hypogea, C. arietinum, V. faba, P. vulgaris, Lupinus albus, Lupinus luteus, Lupinus angustifolius or Lens culinaris. More preferred are the species G. max A. hypogea and M. sativa. Most preferred is the species G. max. When the plant is of the family Solanaceae, the preferred genus is Solanum, Lycopersicon, Nicotiana or Capsicum. Preferred species of the family Solanaceae are S. tuberosum, L. esculentum, N. tabaccum or C. chinense. More preferred is S. tuberosum. Accordingly, in one embodiment the plant is of the family Brassicaceae, preferably of the genus Brassica or Raphanus. Preferred species of the family Brassicaceae are the species B. napus, B. oleracea, B. juncea or B. rapa. More preferred is the species B. napus. When the plant is of the family Chenopodiaceae, the preferred genus is Beta and the preferred species is the B. vulgaris. When the plant is of the family Asteraceae, the preferred genus is Helianthus and the preferred species is H. annuus. When the plant is of the family Malvaceae, the preferred genus is Gossypium or Abelmoschus. When the genus is Gossypium, the preferred species is G. hirsutum or G. barbadense and the most preferred species is G. hirsutum. A preferred species of the genus Abelmoschus is the species A. esculentus. When the plant is of the family Linacea, the preferred genus is Linum and the preferred species is L. usitatissimum. When the plant is of the family Euphorbiaceae, the preferred genus is Manihot, Jatropa or Rhizinus and the preferred species are M. esculenta, J. curcas or R. comunis. When the plant is of the family Convolvulaceae, the preferred genus is Ipomea and the preferred species is I. batatas. When the plant is of the family Rosaceae, the preferred genus is Rosa, Malus, Pyrus, Prunus, Rubus, Ribes, Vaccinium or Fragaria and the preferred species is the hybrid Fragaria×ananassa. When the plant is of the family Cucurbitaceae, the preferred genus is Cucumis, Citrullus or Cucurbita and the preferred species is Cucumis sativus, Citrullus lanatus or Cucurbita pepo. When the plant is of the family Theaceae, the preferred genus is Camellia and the preferred species is C. sinensis. When the plant is of the family Rubiaceae, the preferred genus is Coffea and the preferred species is C. arabica or C. canephora. When the plant is of the family Sterculiaceae, the preferred genus is Theobroma and the preferred species is T. cacao. When the plant is of the genus Citrus, the preferred species is C. sinensis, C. limon, C. reticulata, C. maxima and hybrids of Citrus species, or the like. In a preferred embodiment of the invention, the plant is a soybean, a potato or a corn plant

Suitable methods for transforming or transfecting host cells including plant cells are well known in the art of plant biotechnology. Any method may be used to transform the recombinant expression vector into plant cells to yield the transgenic plants of the invention. General methods for transforming dicotyledenous plants are disclosed, for example, in U.S. Pat. Nos. 4,940,838; 5,464,763, and the like. Methods for transforming specific dicotyledenous plants, for example, cotton, are set forth in U.S. Pat. Nos. 5,004,863; 5,159,135; and 5,846,797. Soybean transformation methods are set forth in U.S. Pat. Nos. 4,992,375; 5,416,011; 5,569,834; 5,824,877; 6,384,301 and in EP 0301749B1 may be used. Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm M E et al., Bio/Technology. 8(9):833-9, 1990; Gordon-Kamm et al. Plant Cell 2:603, 1990), electroporation, incubation of dry embryos in DNAcomprising solution, and microinjection. In the case of these direct transformation methods, the plasmids used need not meet any particular requirements. Simple plasmids, such as those of the pUC series, pBR322, M13mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229. The Agrobacterium-mediated transformation is best suited to dicotyledonous plants but has also been adapted to monocotyledonous plants. The transformation of plants by Agrobacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225. Transformation may result in transient or stable transformation and expression. Although a nucleotide sequence of the present invention can be inserted into any plant and plant cell falling within these broad classes, it is particularly useful in crop plant cells.

The transgenic plants of the invention may be crossed with similar transgenic plants or with transgenic plants lacking the nucleic acids of the invention or with non-transgenic plants, using known methods of plant breeding, to prepare seeds. Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The crossed fertile transgenic plant may have the particular expression cassette inherited through a female parent or through a male parent. The second plant may be an inbred plant. The crossed fertile transgenic may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. Multiple genes in plants or target pathogen species can be down-regulated by gene silencing mechanisms, specifically RNAi, by using a single transgene targeting multiple linked partial sequences of interest. Stacked, multiple genes under the control of individual promoters can also be over-expressed to attain a desired single or multiple phenotype. Constructs containing gene stacks of both over-expressed genes and silenced targets can also be introduced into plants yielding single or multiple agronomically important phenotypes. In certain embodiments the nucleic acid sequences of the present invention can be stacked with any combination of polynucleotide sequences of interest to create desired phenotypes. The combinations can produce plants with a variety of trait combinations including but not limited to disease resistance, herbicide tolerance, yield enhancement, cold and drought tolerance. These stacked combinations can be created by any method including but not limited to cross breeding plants by conventional methods or by genetic transformation. If the traits are stacked by genetic transformation, the polynucleotide sequences of interest can be combined sequentially or simultaneously in any order. For example if two genes are to be introduced, the two sequences can be contained in separate transformation cassettes or on the same transformation cassette. The expression of the sequences can be driven by the same or different promoters.

In accordance with this embodiment, the transgenic plant of the invention is produced by a method comprising the steps of providing a parasitic nematode sca1-like target gene, preparing an expression cassette having a first region that is substantially identical to a portion of the selected sca1-like gene and a second region which is complementary to the first region, transforming the expression cassette into a plant, and selecting progeny of the trans-formed plant which express the dsRNA construct of the invention.

As increased resistance to nematode infection is a general trait wished to be inherited into a wide variety of plants. Increased resistance to nematode infection is a general trait wished to be inherited into a wide variety of plants. The present invention may be used to reduce crop destruction by any plant parasitic nematode. Preferably, the parasitic nematodes belong to nematode families inducing giant or syncytial cells. Nematodes inducing giant or syncytial cells are found in the families Longidoridae, Trichodoridae, Heterodidae, Meloidogynidae, Pratylenchidae or Tylenchulidae. In particular in the families Heterodidae and Meloidogynidae.

Accordingly, parasitic nematodes targeted by the present invention belong to one or more genus selected from the group of Naccobus, Cactodera, Dolichodera, Globodera, Heterodera, Punctodera, Longidorus or Meloidogyne. In a preferred embodiment the parasitic nematodes belong to one or more genus selected from the group of Naccobus, Cactodera, Dolichodera, Globodera, Heterodera, Punctodera or Meloidogyne. In a more preferred embodiment the parasitic nematodes belong to one or more genus selected from the group of Globodera, Heterodera, or Meloidogyne. In an even more preferred embodiment the parasitic nematodes belong to one or both genus selected from the group of Globodera or Heterodera. In another embodiment the parasitic nematodes belong to the genus Meloidogyne.

When the parasitic nematodes are of the genus Globodera, the species are preferably from the group consisting of G. achilleae, G. artemisiae, G. hypolysi, G. mexicana, G. millefolii, G. mali, G. pallida, G. rostochiensis, G. tabacum, and G. virginiae. In another preferred embodiment the parasitic Globodera nematodes includes at least one of the species G. pallida, G. tabacum, or G. rostochiensis. When the parasitic nematodes are of the genus Heterodera, the species may be preferably from the group consisting of H. avenae, H. carotae, H. ciceri, H. cruciferae, H. delvii, H. elachista, H. filipjevi, H. gambiensis, H. glycines, H. goettingiana, H. graduni, H. humuli, H. hordecalis, H. latipons, H. major, H. medicaginis, H. oryzicola, H. pakistanensis, H. rosii, H. sacchari, H. schachtii, H. sorghi, H. trifolii, H. urticae, H. vigni and H. zeae. In another preferred embodiment the parasitic Heterodera nematodes include at least one of the species H. glycines, H. avenae, H. cajani, H. gottingiana, H. trifolii, H. zeae or H. schachtii. In a more preferred embodiment the parasitic nematodes includes at least one of the species H. glycines or H. schachtii. In a most preferred embodiment the parasitic nematode is the species H. glycines. When the parasitic nematodes are of the genus Meloidogyne, the parasitic nematode may be selected from the group consisting of M. acronea, M. arabica, M. arenaria, M. artiellia, M. brevicauda, M. camelliae, M. chitwoodi, M. cofeicola, M. esigua, M. graminicola, M. hapla, M. incognita, M. indica, M. inornata, M. javanica, M. lini, M. mali, M. microcephala, M. microtyla, M. naasi, M. salasi and M. thamesi. In a preferred embodiment the parasitic nematodes includes at least one of the species M. javanica, M. incognita, M. hapla, M. arenaria or M. chitwoodi.

The following examples are not intended to limit the scope of the claims to the invention, but are rather intended to be exemplary of certain embodiments. Any variations in the exemplified methods that occur to the skilled artisan are intended to fall within the scope of the present invention.

### Example 1

### Identification and Isolation of H. glycines Sca1-Like Target Gene

Using total RNA isolated from SCN J2 stage, RT-PCR was used to isolate cDNA fragments that were approximately 400-500 bp in length. The PCR products were cloned into TOPO pCR2.1 vector (Invitrogen, Carlsbad, Calif.) and inserts were confirmed by sequencing. RT-PCR was performed using primer sets (SEQ ID NOs:2 and 3). Briefly, total RNA was isolated from SCN J2 (race 3) using standard TRIzol method (e.g., TriReagent, Molecular Research Center, Inc., Cincinnati, Ohio). RT-PCR reactions contained SCN J2 total RNA. A gene fragment represented by nucleotides 1-499 of SEQ ID NO:1 was isolated using this method, and determined to be a homolog of C. elegans sca1.

In order to obtain full-length cDNA for H. glycines sca1-like, an RT-PCT method, based on highly conserved spliced leader sequence (SL1) present in many nematode species, is used. The reactions are conducted using Supercript One-Step kit (Invitrogen, Carlsbad, Calif., catalog no. 10928-034) and a primer set. The forward primer is a 22-mer SL1 sequence (SEQ ID NO:13) and reverse primers will be gene specific and are located in the previously cloned cDNA region. PCR products will be cloned into Pcr4-topo VECTOR (Invitrogen, Carlsbad, Calif.) and sequenced.

3′cDNA ends were amplified using the GeneRacer Kit (Invitrogen, Carlsbad, Calif., catalog No. L1500-01). The first-strand cDNAs were generated through reverse transcription using total RNA and the GeneRacer Oligo dT Primer (SEQ ID NO:12). The 3′ RACE PCR was performed with the GeneRacer 3′ Primer (SEQ ID NO:5) and a gene-specific forward primer (SEQ ID NO:4). The nested PCR reactions were subsequently conducted using GeneRacer 3′ Nested Primer (SEQ ID NO:7) and a gene-specific forward primer (SEQ ID NO:6). PCR products were cloned into pCR4-TOPO (Invitrogen, Carlsbad, Calif.) and sequenced.

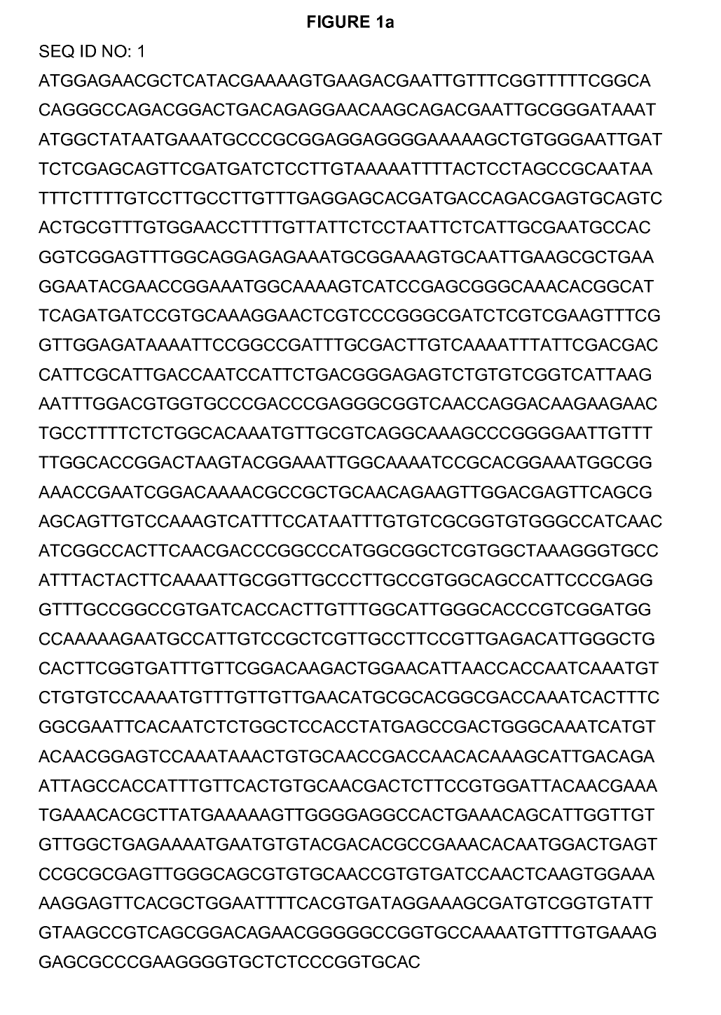

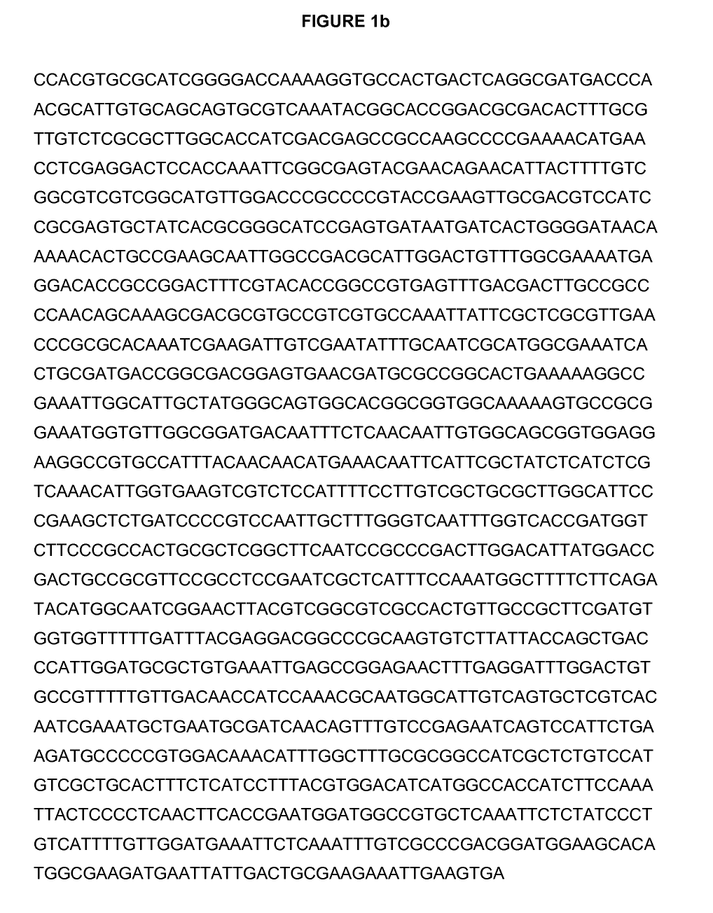

The sequences of the sca1-like PCR fragments isolated above were assembled into cDNA corresponding to the gene designated H. glycines sca1-like, and this sequence is set forth as SEQ ID NO: 1 in FIG. 1.

### Example 2

### Demonstration of Essentiality of C. elegans Target Gene and Isolation of Homologs from SCN

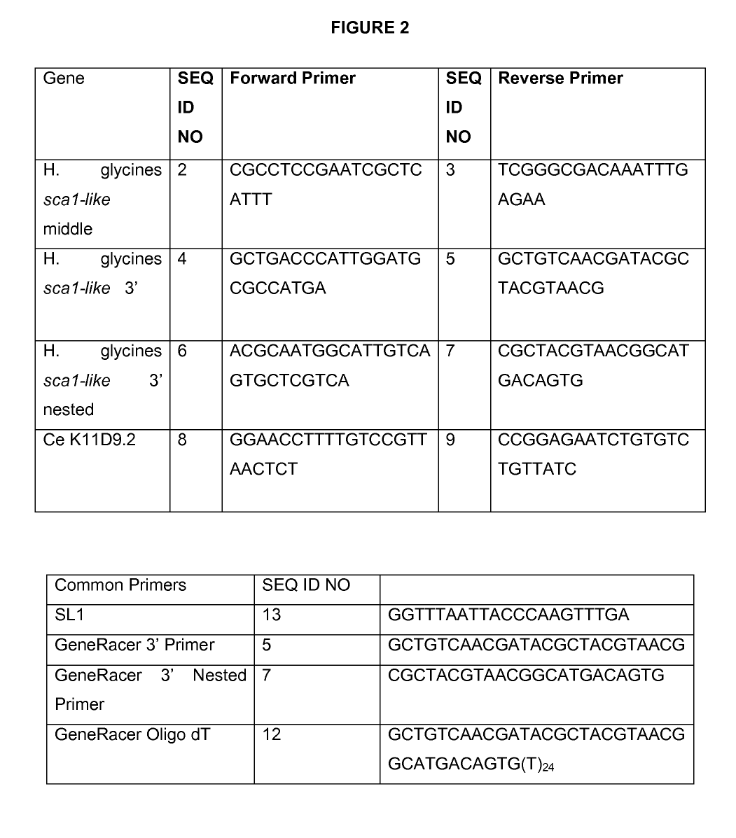

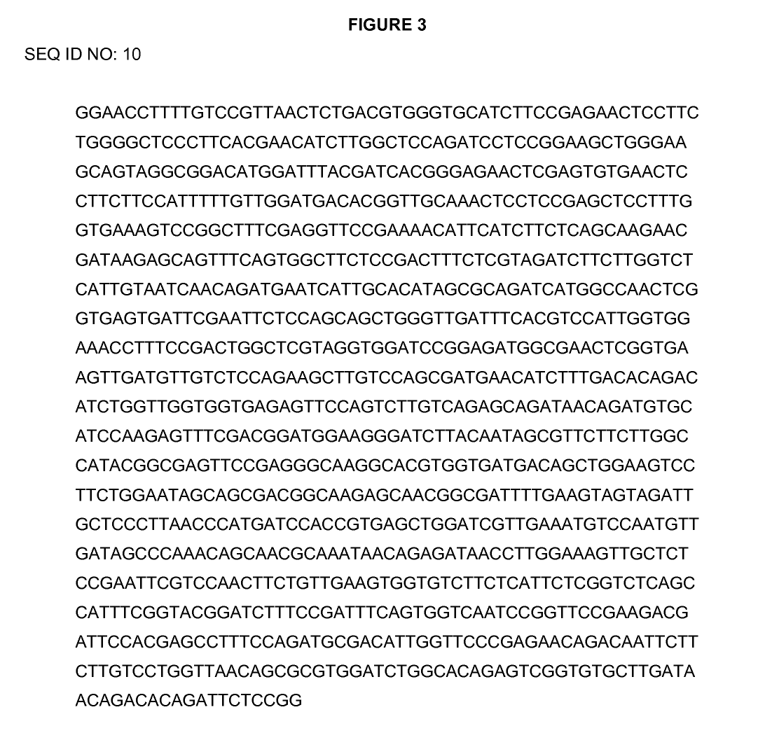

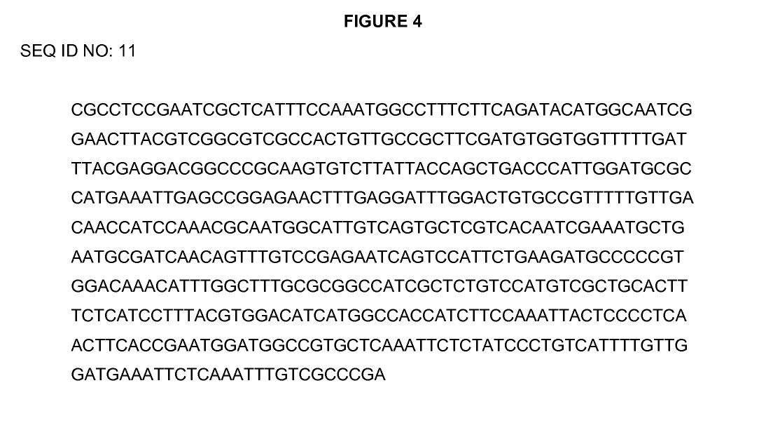

The homolog of the SCN target gene identified in Example 1 was isolated from C. elegans using PCR primers (SEQ ID NOs: 8 and 9 in FIG. 2) and C. elegans genomic DNA as a template. (see K11D9.2, Genbank, National Center for Biotechnology Information, Bethesda, Md.) The PCR products (˜1 kb in length) were cloned into the multiple cloning site of pLitmus28i (New England Biolabs, Beverly, Mass.), so that C. elegans gene fragments were flanked by two T7 promoters in a head-to-head configuration. The DNA sequences of C. elegans gene fragment used in RNAi assay are shown in FIG. 3 (SEQ ID NO:10).

The pLitmus28i vectors with the target genes were then transformed into E. coli strain HT115(DE3). This strain is deficient in RNase III—an enzyme that degrades dsRNA. Therefore, dsRNA produced in HT115(DE3) is expected to be more stable. Upon IPTG (Isopropyl β-D-Thiogalactopyranoside) induction, T7 RNA polymerase, was expressed and transcribed dsRNA. The production of dsRNA in E. coli was confirmed by total RNA extraction using RiboPure-Bacteria Kit (Ambion, Austin, Tex., cat no 1925) and subsequent S1 nuclease treatment.

The C. elegans RNAi feeding assay consisted growing the HT115(DE3) cultures overnight and adding 50 μl of the E. coli cultures to each well of a 96 well microtiter plate, Approximately 3 μl of L1 larvae (10 to 15 L1s) were then added to each well, and the plate was incubated at approximately 25° C. for 5 days. Each culture was triplicated, so a total of six wells were used for each C. elegans gene tested in the assay. The bacteria transformed with pLitmus28i alone (no inserts) was used as the control. The assay was examined and RNAi phenotypes of the C. elegans were analyzed.

By Day 5, in the control (pLitmus28i alone), L1 larvae developed into gravid adults and produced many progeny. The administration by feeding dsRNA substantially identical to the C. elegans target gene resulted in arrest in development of nematodes, and the worms in all six wells for the gene showed consistent RNAi phenotypes. A dsRNA substantially identical to the C. elegans sca1 gene (SEQ ID NO:10), the homolog of H. glycines sca1-like (SEQ ID NO:1)), caused mortality of the adult as evidenced by a phenotype of a rigid, non-moving straight body type rather than the living plant, moving s-shaped body type. These data demonstrated that C. elegans homologue of the sca1-like target gene candidate identified in Example 1 is essential for C. elegans development. This further indicated that the selected target gene indeed plays a key role for nematode survival in both plant parasitic nematodes and C. elegans.

### Example 3

### Binary Vector Construction for Soybean Transformation

This exemplified method employs a binary vector containing the sca1-like target gene. The vector consists of an antisense fragment (SEQ ID NO:11) of the target sca1-like gene, a spacer, a sense fragment of the target gene and a vector backbone. The sequence of the sca1-like gene (SEQ ID NO.1) is set forth in FIG. 1. The target gene fragment (SEQ ID NO:11) corresponding to nucleotides 1-499 of SEQ ID NO:1 was used to construct the binary vector RSA006 (pSA006). In this vector, dsRNA for the sca1-like target gene was expressed under a constitutive promoter, Super Promoter (see U.S. Pat. No. 5,955,646, incorporated herein by reference). The selection marker for transformation was a mutated AHAS gene from Arabidopsis thaliana that conferred resistance to the herbicide ARSENAL (imazepyr, BASF Corporation, Mount Olive, N.J.). The expression of mutated AHAS was driven by a ubiquitin promoter. (See Plesch, G. and Ebneth, M., “Method for the stable expression of nucleic acids in transgenic plants, controlled by a parsley ubiquitin promoter”, WO 03/102198, hereby incorporated by reference.)

### Example 4

### Bioassay of dsRNA Targeted to H. glycines sca1 Target Gene

The rooted explant assay was employed to demonstrate dsRNA expression and the resulting nematode resistance. This assay can be found in co-pending application U.S. Ser. No. 12/001,234, the contents of which are incorporated herein by reference.

Clean soybean seeds from soybean cultivar were surface sterilized and germinated. Three days before inoculation, an overnight liquid culture of the disarmed Agrobacterium culture, for example, the disarmed A. rhizogenes strain K599 containing the binary vector RSA006, was initiated. The next day the culture was spread onto an LB agar plate containing kanamycin as a selection agent. The plates were incubated at 28° C. for two days. One plate was prepared for every 50 explants to be inoculated. Cotyledons containing the proximal end from its connection with the seedlings were used as the explant for transformation. After removing the cotyledons the surface was scraped with a scalpel around the cut site. The cut and scraped cotyledon was the target for Agrobacterium inoculation. The prepared explants were dipped onto the disarmed thick A. rhizogenes colonies prepared above so that the colonies were visible on the cut and scraped surface. The explants were then placed onto 1% agar in Petri dishes for co-cultivation under light for 6-8 days.

After the transformation and co-cultivation soybean explants were transferred to rooting induction medium with a selection agent, for example S-B5-708 for the mutated acetohydroxy acid synthase (AHAS) gene (Sathasivan et al., Plant Phys. 97:1044-50, 1991). Cultures were maintained in the same condition as in the co-cultivation step. The S-B5-708 medium comprises: 0.5×B5 salts, 3 mM MES, 2% sucrose, 1×B5 vitamins, 400 μg/ml Timentin, 0.8% Noble agar, and 1 μM Imazapyr (selection agent for AHAS gene) (BASF Corporation, Florham Park, N.J.) at pH5.8.

Two to three weeks after the selection and root induction, transformed roots were formed on the cut ends of the explants. Explants were transferred to the same selection medium (S-B5-708 medium) for further selection. Transgenic roots proliferated well within one week in the medium and were ready to be subcultured.

Strong and white soybean roots were excised from the rooted explants and cultured in root growth medium supplemented with 200 mg/l Timentin (S-MS-606 medium) in six-well plates. Cultures were maintained at room temperature under the dark condition. The S-MS-606 medium comprises: 0.2×MS salts and B5 vitamins, 2% sucrose, and 200 mg/l Timentin at pH5.8.

One to five days after subculturing, the roots were inoculated with surface sterilized nematode juveniles in multi-well plates for either gene of interest or promoter construct assay. As a control, soybean cultivar Williams 82 control vector and Jack control vector roots were used. The root cultures of each line that occupied at least half of the well were inoculated with surface-decontaminated race 3 of soybean cyst nematode (SCN) second stage juveniles (J2) at the level of 500 J2/well. The plates were then sealed and put back into the incubator at 25° C. in darkness. Several independent root lines were generated from each binary vector transformation and the lines were used for bioassay. Four weeks after nematode inoculation, the cysts in each well were counted. Bioassay results for construct RSA006 show a statistically significant reduction (p-value <0.05) in cyst count over multiple transgenic lines and a general trend of reduced cyst count in the majority of transgenic lines tested.

Those skilled in the art will recognize, or will be able to ascertain using no more than routine experimentation, many equivalents to the specific embodiments of the invention described herein. Such equivalents are intended to be encompassed by the following claims.

1. A dsRNA molecule comprising a) a first strand comprising a sequence substantially identical to a portion of a sca1-like target gene of a parasitic nematode, and b) a second strand comprising a sequence substantially complementary to the first strand, wherein the target gene is a parasitic nematode sca1-like gene.

2. The dsRNA molecule of claim 1, wherein the portion of the target gene is of a sequence selected from the group consisting of: a) a polynucleotide comprising a sequence as set forth in SEQ ID NO:1, 10 or 11; b) a polynucleotide comprising a sequence having at least 80% sequence identity to SEQ ID NO.1, 10 or 11; and c) a polynucleotide from a nematode that hybridizes under stringent conditions to a polynucleotide comprising a sequence as set forth in SEQ ID NO:1, 10 or 11.

3. A pool of dsRNA molecules comprising a multiplicity of RNA molecules each comprising a double stranded region having a length of about 19 to 24 nucleotides, wherein said RNA molecules are derived from a polynucleotide selected from the group consisting of: a) a polynucleotide comprising a sequence as set forth in SEQ ID NO:1, 10 or 11; b) a polynucleotide comprising a sequence having at least 80% sequence identity to SEQ ID NO.1, 10 or 11; and c) a polynucleotide from a nematode that hybridizes under stringent conditions to a polynucleotide comprising a sequence as set forth in SEQ ID NO:1, 10 or 11.

4. A transgenic plant capable of expressing a dsRNA that is substantially identical to a portion of a parasitic nematode sca1-like target gene.

5. The transgenic plant of claim 4, wherein the target gene comprises a sequence selected from the group consisting of: a) a polynucleotide comprising a sequence as set forth in SEQ ID NO:1, 10 or 11; b) a polynucleotide comprising a sequence having at least 80% sequence identity to SEQ ID NO.1, 10 or 11; and c) a polynucleotide from a parasitic nematode that hybridizes under stringent conditions to a polynucleotide comprising a sequence as set forth in SEQ ID NO:1, 10 or 11.

6. The transgenic plant of claim 4, wherein the dsRNA comprises a multiplicity of RNA molecules each comprising a double stranded region having a length of about 19-24 nucleotides, wherein said RNA molecules are derived a polynucleotide selected from the group consisting of: a) a polynucleotide comprising a sequence as set forth in SEQ ID NO:1, 10 or 11; b) a polynucleotide comprising a sequence having at least 80% sequence identity to SEQ ID NO.1, 10 or 11; and c) a polynucleotide from a parasitic nematode that hybridizes under stringent conditions to a polynucleotide comprising a sequence as set forth in SEQ ID NO:1, 10 or 11.

7. The transgenic plant of claim 4, wherein the plant is selected from the group consisting of: soybean, potato, tomato, peanuts, cotton, cassaya, coffee, coconut, pineapple, citrus trees, banana, corn, rape, beet, sunflower, sorghum, wheat, oats, rye, barley, rice, green bean, lima bean, pea, and tobacco.

8. The transgenic plant of claim 4 wherein the plant is a soybean plant.

9. A method for controlling the infection of a plant by a parasitic nematode, comprising the steps of exposing the nematode to a dsRNA molecule that is substantially identical to a portion of a target gene essential to the nematode, thereby controlling the infection of the plant by the nematode, wherein the target gene a parasitic nematode sca1-like gene.

10. The method of claim 9, wherein the target gene comprises a sequence selected from the group consisting of: a) a polynucleotide comprising a sequence as set forth in SEQ ID NO:1, 10 or 11; b) a polynucleotide comprising a sequence having at least 80% sequence identity to SEQ ID NO.1, 10 or 11; and c) a polynucleotide from a parasitic nematode that hybridizes under stringent conditions to a polynucleotide comprising a sequence as set forth in SEQ ID NO:1, 10 or 11.

11. A method of making a transgenic plant capable of expressing a sca1-like dsRNA that is substantially identical to a portion of a target gene in a parasitic nematode, said method comprising the steps of: a) preparing a nucleic acid sequence having a region that is substantially identical to a portion of a parasitic nematode sca1-like target gene, wherein the nucleic acid is able to form a sca1-like double-stranded transcript once expressed in the plant; b) transforming a recipient plant with said nucleic acid; c) producing one or more transgenic offspring of said recipient plant; and d) selecting the offspring for expression of said transcript.

12. The method of claim 11, wherein the target gene comprises a sequence selected from the group consisting of: a) a polynucleotide comprising a sequence as set forth in SEQ ID NO:1, 10 or 11; b) a polynucleotide comprising a sequence having at least 80% sequence identity to SEQ ID NO.1, 10 or 11; and c) a polynucleotide from a parasitic nematode that hybridizes under stringent conditions to a polynucleotide comprising a sequence as set forth in SEQ ID NO:1, 10 or 11.

13. The method of claim 11, wherein the portion of the target gene is from about 19 to about 400 nucleotides of a sequence selected from the group consisting of: a) a polynucleotide comprising a sequence as set forth in SEQ ID NO:1, 10 or 11; and b) a polynucleotide from a parasitic nematode that hybridizes under stringent conditions to a polynucleotide comprising a sequence as set forth in SEQ ID NO:1, 10 or 11.

14. The method of claim 11, wherein the plant is selected from the group consisting of: soybean, potato, tomato, peanuts, cotton, cassaya, coffee, coconut, pineapple, citrus trees, banana, corn, rape, beet, sunflower, sorghum, wheat, oats, rye, barley, rice, green bean, lima bean, pea, and tobacco.

15. The method of claim 11 wherein the plant is a soybean plant.


SEQUENCE LISTING (13 sequences)

SEQ ID NO: 1 - DNA, 3033 bp, Heterodera glycines
atggagaacgctcatacgaaaagtgaagacgaattgtttcggtttttcggcacagggccagacggactgacagaggaacaagcagacgaattgcgggataaatatggctataatgaaatgcccgcggaggaggggaaaaagctgtgggaattgattctcgagcagttcgatgatctccttgtaaaaattttactcctagccgcaataatttcttttgtccttgccttgtttgaggagcacgatgaccagacgagtgcagtcactgcgtttgtggaaccttttgttattctcctaattctcattgcgaatgccacggtcggagtttggcaggagagaaatgcggaaagtgcaattgaagcgctgaaggaatacgaaccggaaatggcaaaagtcatccgagcgggcaaacacggcattcagatgatccgtgcaaaggaactcgtcccgggcgatctcgtcgaagtttcggttggagataaaattccggccgatttgcgacttgtcaaaatttattcgacgaccattcgcattgaccaatccattctgacgggagagtctgtgtcggtcattaagaatttggacgtggtgcccgacccgagggcggtcaaccaggacaagaagaactgccttttctctggcacaaatgttgcgtcaggcaaagcccggggaattgtttttggcaccggactaagtacggaaattggcaaaatccgcacggaaatggcggaaaccgaatcggacaaaacgccgctgcaacagaagttggacgagttcagcgagcagttgtccaaagtcatttccataatttgtgtcgcggtgtgggccatcaacatcggccacttcaacgacccggcccatggcggctcgtggctaaagggtgccatttactacttcaaaattgcggttgcccttgccgtggcagccattcccgagggtttgccggccgtgatcaccacttgtttggcattgggcacccgtcggatggccaaaaagaatgccattgtccgctcgttgccttccgttgagacattgggctgcacttcggtgatttgttcggacaagactggaacattaaccaccaatcaaatgtctgtgtccaaaatgtttgttgttgaacatgcgcacggcgaccaaatcactttcggcgaattcacaatctctggctccacctatgagccgactgggcaaatcatgtacaacggagtccaaataaactgtgcaaccgaccaacacaaagcattgacagaattagccaccatttgttcactgtgcaacgactcttccgtggattacaacgaaatgaaacacgcttatgaaaaagttggggaggccactgaaacagcattggttgtgttggctgagaaaatgaatgtgtacgacacgccgaaacacaatggactgagtccgcgcgagttgggcagcgtgtgcaaccgtgtgatccaactcaagtggaaaaaggagttcacgctggaattttcacgtgataggaaagcgatgtcggtgtattgtaagccgtcagcggacagaacgggggccggtgccaaaatgtttgtgaaaggagcgcccgaaggggtgctctcccggtgcacccacgtgcgcatcggggaccaaaaggtgccactgactcaggcgatgacccaacgcattgtgcagcagtgcgtcaaatacggcaccggacgcgacactttgcgttgtctcgcgcttggcaccatcgacgagccgccaagccccgaaaacatgaacctcgaggactccaccaaattcggcgagtacgaacagaacattacttttgtcggcgtcgtcggcatgttggacccgccccgtaccgaagttgcgacgtccatccgcgagtgctatcacgcgggcatccgagtgataatgatcactggggataacaaaaacactgccgaagcaattggccgacgcattggactgtttggcgaaaatgaggacaccgccggactttcgtacaccggccgtgagtttgacgacttgccgccccaacagcaaagcgacgcgtgccgtcgtgccaaattattcgctcgcgttgaacccgcgcacaaatcgaagattgtcgaatatttgcaatcgcatggcgaaatcactgcgatgaccggcgacggagtgaacgatgcgccggcactgaaaaaggccgaaattggcattgctatgggcagtggcacggcggtggcaaaaagtgccgcggaaatggtgttggcggatgacaatttctcaacaattgtggcagcggtggaggaaggccgtgccatttacaacaacatgaaacaattcattcgctatctcatctcgtcaaacattggtgaagtcgtctccattttccttgtcgctgcgcttggcattcccgaagctctgatccccgtccaattgctttgggtcaatttggtcaccgatggtcttcccgccactgcgctcggcttcaatccgcccgacttggacattatggaccgactgccgcgttccgcctccgaatcgctcatttccaaatggcttttcttcagatacatggcaatcggaacttacgtcggcgtcgccactgttgccgcttcgatgtggtggtttttgatttacgaggacggcccgcaagtgtcttattaccagctgacccattggatgcgctgtgaaattgagccggagaactttgaggatttggactgtgccgtttttgttgacaaccatccaaacgcaatggcattgtcagtgctcgtcacaatcgaaatgctgaatgcgatcaacagtttgtccgagaatcagtccattctgaagatgcccccgtggacaaacatttggctttgcgcggccatcgctctgtccatgtcgctgcactttctcatcctttacgtggacatcatggccaccatcttccaaattactcccctcaacttcaccgaatggatggccgtgctcaaattctctatccctgtcattttgttggatgaaattctcaaatttgtcgcccgacggatggaagcacatggcgaagatgaattattgactgcgaagaaattgaagtga

SEQ ID NO: 2 - DNA, 20 bp, Heterodera glycines
cgcctccgaatcgctcattt

SEQ ID NO: 3 - DNA, 20 bp, Heterodera glycines
tcgggcgacaaatttgagaa

SEQ ID NO: 4 - DNA, 24 bp, Heterodera glycines
gctgacccattggatgcgccatga

SEQ ID NO: 5 - DNA, 25 bp, Artificial
  primer
gctgtcaacgatacgctacgtaacg

SEQ ID NO: 6 - DNA, 27 bp, Heterodera glycines
acgcaatggcattgtcagtgctcgtca

SEQ ID NO: 7 - DNA, 23 bp, Artificial
  primer
cgctacgtaacggcatgacagtg

SEQ ID NO: 8 - DNA, 23 bp, Caenorhabditis elegans
ggaaccttttgtccgttaactct

SEQ ID NO: 9 - DNA, 23 bp, Caenorhabditis elegans
ccggagaatctgtgtctgttatc

SEQ ID NO: 10 - DNA, 1063 bp, Caenorhabditis elegans
ggaaccttttgtccgttaactctgacgtgggtgcatcttccgagaactccttctggggctcccttcacgaacatcttggctccagatcctccggaagctgggaagcagtaggcggacatggatttacgatcacgggagaactcgagtgtgaactccttcttccatttttgttggatgacacggttgcaaactcctccgagctcctttggtgaaagtccggctttcgaggttccgaaaacattcatcttctcagcaagaacgataagagcagtttcagtggcttctccgactttctcgtagatcttcttggtctcattgtaatcaacagatgaatcattgcacatagcgcagatcatggccaactcggtgagtgattcgaattctccagcagctgggttgatttcacgtccattggtggaaacctttccgactggctcgtaggtggatccggagatggcgaactcggtgaagttgatgttgtctccagaagcttgtccagcgatgaacatctttgacacagacatctggttggtggtgagagttccagtcttgtcagagcagataacagatgtgcatccaagagtttcgacggatggaagggatcttacaatagcgttcttcttggccatacggcgagttccgagggcaaggcacgtggtgatgacagctggaagtccttctggaatagcagcgacggcaagagcaacggcgattttgaagtagtagattgctcccttaacccatgatccaccgtgagctggatcgttgaaatgtccaatgttgatagcccaaacagcaacgcaaataacagagataaccttggaaagttgctctccgaattcgtccaacttctgttgaagtggtgtcttctcattctcggtctcagccatttcggtacggatctttccgatttcagtggtcaatccggttccgaagacgattccacgagcctttccagatgcgacattggttcccgagaacagacaattcttcttgtcctggttaacagcgcgtggatctggcacagagtcggtgtgcttgataacagacacagattctccgg

SEQ ID NO: 11 - DNA, 499 bp, Heterodera glycines
cgcctccgaatcgctcatttccaaatggcctttcttcagatacatggcaatcggaacttacgtcggcgtcgccactgttgccgcttcgatgtggtggtttttgatttacgaggacggcccgcaagtgtcttattaccagctgacccattggatgcgccatgaaattgagccggagaactttgaggatttggactgtgccgtttttgttgacaaccatccaaacgcaatggcattgtcagtgctcgtcacaatcgaaatgctgaatgcgatcaacagtttgtccgagaatcagtccattctgaagatgcccccgtggacaaacatttggctttgcgcggccatcgctctgtccatgtcgctgcactttctcatcctttacgtggacatcatggccaccatcttccaaattactcccctcaacttcaccgaatggatggccgtgctcaaattctctatccctgtcattttgttggatgaaattctcaaatttgtcgcccga

SEQ ID NO: 12 - DNA, 37 bp, Artificial
  primer
gctgtcaacgatacgctacgtaacggcatgacagtgt

SEQ ID NO: 13 - DNA, 21 bp, Artificial
  primer
ggtttaattacccaagtttga